SEQUENCE LISTING (124 sequences)

SEQ ID NO: 1 - DNA, 1053 bp, Escherichia coli W3110
atgaattatcagaacgacgatttacgcatcaaagaaatcaaagagttacttcctcctgtcgcattgctggaaaaattccccgctactgaaaatgccgcgaatacggttgcccatgcccgaaaagcgatccataagatcctgaaaggtaatgatgatcgcctgttggttgtgattggcccatgctcaattcatgatcctgtcgcggcaaaagagtatgccactcgcttgctggcgctgcgtgaagagctgaaagatgagctggaaatcgtaatgcgcgtctattttgaaaagccgcgtaccacggtgggctggaaagggctgattaacgatccgcatatggataatagcttccagatcaacgacggtctgcgtatagcccgtaaattgctgcttgatattaacgacagcggtctgccagcggcaggtgagtttctcgatatgatcaccccacaatatctcgctgacctgatgagctggggcgcaattggcgcacgtaccaccgaatcgcaggtgcaccgcgaactggcatcagggctttcttgtccggtcggcttcaaaaatggcaccgacggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcacaggtgatgatcgatttcagccatgctaactcgtccaaacaattcaaaaagcagatggatgtttgtgctgacgtttgccagcagattgccggtggcgaaaaggccattattggcgtgatggtggaaagccatctggtggaaggcaatcagagcctcgagagcggggagccgctggcctacggtaagagcatcaccgatgcctgcatcggctgggaagataccgatgctctgttacgtcaactggcgaatgcagtaaaagcgcgtcgcgggtaa

SEQ ID NO: 2 - protein, 350 aa, Escherichia coli W3110
MNYQNDDLRIKEIKELLPPVALLEKFPATENAANTVAHARKAIHKILKGNDDRLLVVIGPCSIHDPVAAKEYATRLLALREELKDELEIVMRVYFEKPRTTVGWKGLINDPHMDNSFQINDGLRIARKLLLDINDSGLPAAGEFLDMITPQYLADLMSWGAIGARTTESQVHRELASGLSCPVGFKNGTDGTIKVAIDAINAAGAPHCFLSVTKWGHSAIVNTSGNGDCHIILRGGKEPNYSAKHVAEVKEGLNKAGLPAQVMIDFSHANSSKQFKKQMDVCADVCQQIAGGEKAIIGVMVESHLVEGNQSLESGEPLAYGKSITDACIGWEDTDALLRQLANAVKARRG

SEQ ID NO: 3 - DNA, 49 bp, Artificial Sequence
  fhuA arm 1_F
ccaagcgacgcccaacctgccatcatggcgcgttccaaaactgctcagc

SEQ ID NO: 4 - DNA, 40 bp, Artificial Sequence
  fhuA arm 1_R
tatcctcgagactagtgagctcctttcagcggttcggtgg

SEQ ID NO: 5 - DNA, 40 bp, Artificial Sequence
  fhuA arm 2_F
ctagtctcgaggatatcgaccacaccctgctgaccggtgt

SEQ ID NO: 6 - DNA, 48 bp, Artificial Sequence
  fhuA arm 2_R
atagaataccaattggcatgctttttagaaacggaaggttgcggttgc

SEQ ID NO: 7 - DNA, 34 bp, Artificial Sequence
  Pn_aroG_F
aaaggagctcactagtaggatgctcctgttatgg

SEQ ID NO: 8 - DNA, 36 bp, Artificial Sequence
  Pn_aroG_R
cagggtgtggtcgatatcttacccgcgacgcgcttt

SEQ ID NO: 9 - DNA, 34 bp, Artificial Sequence
  Ptrc_F
aaaggagctcactagtccgcttgctgcaactctc

SEQ ID NO: 10 - DNA, 40 bp, Artificial Sequence
  Ptrc_R
tggacgatactcatatgtttcctgtgtgaaattgttatcc

SEQ ID NO: 11 - DNA, 35 bp, Artificial Sequence
  Pcj1_F
aaaggagctcactagtaccgcgggcttattccatt

SEQ ID NO: 12 - DNA, 41 bp, Artificial Sequence
  Pcj1_R
atctatagtctgcatatgttaatctcctagattgggtttca

SEQ ID NO: 13 - DNA, 32 bp, Artificial Sequence
  aroG_F
taggagattaacatatgaattatcagaacgac

SEQ ID NO: 14 - DNA, 18 bp, Artificial Sequence
  certification primer
ggaacgctcagattgcgt

SEQ ID NO: 15 - DNA, 45 bp, Artificial Sequence
  Ava_ABCD_F
acaatttcacacaggaaagatatcatgagtatcgtccaagcaaag

SEQ ID NO: 16 - DNA, 45 bp, Artificial Sequence
  Ava_ABCD_R
ctcatccgccaaaacagctctagattatgaattattttccagaca

SEQ ID NO: 17 - DNA, 6461 bp, Anabaena variabilis ATCC29413
atgagtatcgtccaagcaaagtttgaagctaaggaaacatcttttcatgtagaaggttacgaaaagattgagtatgatttggtgtatgtagatggtatttttgaaatccagaattctgcactagcagatgtatatcaaggttttggacgatgcttggcgattgtagatgctaacgtcagtcggttgtatggtaatcaaattcaggcatatttccagtattatggtatagaactgaggctatttcctattaccattactgaaccagataagactattcaaactttcgagagagttatagatgtctttgcagatttcaaattagtccgcaaagaaccagtattagtcgtgggtggcggtttaattacagatgttgtcggctttgcttgttctacatatcgtcgcagcagcaattacatccgcattcctactacattgattggattaattgatgccagtgtagcaattaaggtagcagttaatcatcgcaaactgaaaaaccgtttgggtgcttatcatgcttctcgcaaagtatttttagatttctccttgttgcgtactctccctacagaccaagtacgtaacgggatggcggaattggtaaaaatcgctgtagtagcgcatcaagaagtttttgaattgttggagaagtacggcgaagaattactacgtactcattttggcaatatagatgcaactccagagattaaagaaatagcccatcgtttgacttacaaagctatccataagatgttggaattggaagttcccaacctgcatgagttagacctagatagggtgattgcttacggtcacacttggagtcccaccttggaacttgcgcctcgtctacccatgttccacggacacgccgttaatgtagatatggctttctcggcaacgatcgccgcccgtagaggatatattacaattgcagaacgcgatcgtattttaggattaatgagtcgcgttggtctatccctcgaccatcccatgttggatatagatattttgtggcgtggtactgaatctatcacattaactcgtgatggtttgttaagagctgctatgccaaaacccattggtgattgtgtcttcgtcaatgacctgacaagagaagaattagcagccgcattagctgaccacaaagaactttgtaccagttatccccgtggtggtgaaggtgtggatgtgtatcccgtttatcaaaaagaattaatcgggagtgttaaataatgacttttttgaattcaaaatgcaaaatactccacggatacactgcgcgagcgcggtagcatttctgttcgcggagcgtcccgtagggaaagagaaggctacgcaaataatcggacactaattgtctttaattttgaattttgaattttgaattttgaattggagcgaagcgacttgacaaatgtgattgtccaaccaacagctagacctgttacaccattgggaattttaaccaagcagttagaagccatagtccaagaggttaagcaacatccagatttacctggggaattgatagcaaacatccatcaggcttggcgtttagccgcaggtatagacccttatttggaagaatgcaccactccagaatctcctgaactcgctgcattggcaaaaaccacagccaccgaagcctggggagaacacttccacggaggtacaaccgtccgtcctctagaacaagagatgctttctggtcatatcgaaggacaaaccttaaagatgtttgttcacatgaccaaagctaaaaaagtcttagaaattgggatgtttaccggttattcggcgctggcgatggcggaagcattaccagaggatggactgcttgtggcttgtgaagttgacccttacgcggcggaaattggacagaaagcctttcaacaatctccccacggtggaaagattcgtgtggaattggatgcagccttagcaactcttgataagttagcagaagctggggagtcttttgacttggtatttatcgacgcagataaaaaagagtatgtagcctattttcacaagttgctaggtagcagtttgttagcaccagatggctttatttgtgtagataacaccttattacaaggggaagtttatctaccagcagaggaacgtagcgtcaatggtgaagcgatcgcgcaatttaatcatacagtagctatagacccccgtgtagaacaggttttgttgccgttgcgagatggtttaacaattatccgcagaatacaaccttaattgtccaatcgactatggcacaatcccttcccctttcttccgcacctgctacaccgtctcttccttcccagacgaaaatagccgcaattatccaaaatatctgcactttggctttgttattactagcattgcccattaatgccaccattgtttttatatccttgttagtcttccgaccgcaaaaggtcaaagcagcaaacccccaaaccattcttatcagtggcggtaagatgaccaaagctttacaactagcaaggtcattccacgcggctggacatagagttgtcttggtggaaacccataaatactggttgactggtcatcgtttttcccaagcagtggataagttttacacagtccccgcaccccaggacaatccccaagcttacattcaggctttggtagatatcgtcaaacaagaaaacatcgatgtttatattcccgtcaccagtccagtgggtagctactacgactcattagccaaaccagagttatcccattattgcgaagtgtttcactttgacgcagatattacccaaatgttggatgataaatttgcgttgacacaaaaagcgcgatcgcttggtttatcagtacccaaatcctttaaaattacctcaccagaacaagtcatcaacttcgatttttctggagagacacgtaaatacatcctcaaaagcattccctacgactcagtgcggcggttggacttaaccaaactcccctgtgctactccagaggaaacagcagcattcgtcagaagtttgccaattactcccgaaaaaccgtggattatgcaggaatttatccccggtaaggaattctgcacccatagcaccgttcggaatggggaactcagactgcattgctgttgcgaatcttcagccttccaagttaattatgagaatgtaaataacccgcaaattaccgaatgggtacagcattttgtcaaggaactgaaactgacaggacagatttcctttgactttatccaagccgaagacggaacagtttacgccatcgagtgtaacccccgcacacattcagcaattaccacattttacgaccacccccaggtagcagaagcgtacttgagtcaagcaccgacgactgaaaccatacaaccactaacgacaagcaagcctacctattggacttatcacgaagtttggcgtttaactggtatccgttctttcacccagttgcaaagatggctggggaatatttggcgcgggactgatgcgatttatcagccagatgaccccttaccgtttttgatggtacatcattggcaaattcccctactgttattgaataatttgcgtcgtcttaaaggttggacgcggatagatttcaatattgggaagttggtggaattggggggagattagtttttaaacgcagagggacgctgaggttagcgcagcgaaaagttctggaggagggtttccctccgtaggaaacttttcaagagagagggacgcggagtgtgttttctctgcgtctctgcgtgagaaattttttattattgagcaaagttagaagatatgcagactatagattttaatattcgtaagttacttgtagagtggaacgcgacccacagagattatgatctttcccagagtttacatgaactaattgtagctcaagtagaacgaacacctgaggcgatcgctgtcacctttgacaagcaacaactaacttatcaagaactaaatcataaagcaaaccagctaggacattatttacaaacattaggagtccagccagaaaccctggtaggcgtttgtttagaacgttccttagaaatggttatctgtcttttaggaatcctcaaagctgggggtgcttatgttcctattgaccctgaatatcctcaagaacgcatagcttatatgctagaagattctcaggtgaaggtactactaactcaagaaaaattactcaatcaaattccccaccatcaagcacaaactatctgtgtagatagggaatgggagaaaatttccacacaagctaataccaatcccaaaagtaatataaaaacggataatcttgcttatgtaatttacacctctggttccactggtaaaccaaaaggtgcaatgaacacccacaaaggtatctgtaatcgcttattgtggatgcaggaagcttatcaaatcgattccacagatagcattttacaaaaaaccccctttagttttgatgtttccgtttgggagttcttttggactttattaactggcgcacgtttggtaatagccaaaccaggcggacataaagatagtgcttacctcatcgatttaattactcaagaacaaatcactacgttgcattttgtcccctcaatgctgcaagtgtttttacaaaatcgccatgtaagcaaatgcagctctctaaaaagagttatttgtagcggtgaagctttatctatagatttacaaaatagatttttccagcatttgcaatgtgaattacataacctctatggcccgacagaagcagcaattgatgtcacattttggcaatgtagaaaagatagtaatttaaagagtgtacctattggtcgtcccattgctaatactcaaatttatattcttgatgccgatttacaaccagtaaatattggtgtcactggtgaaatttatattggtggtgtaggggttgctcgtggttatttgaataaagaagaattgaccaaagaaaaatttattattaatccctttcccaattctgagtttaagcgactttataaaacaggtgatttagctcgttatttacccgatggaaatattgaatatcttggtagaacagattatcaagtaaaaattcggggttatagaattgaaattggcgagattgaaaatgttttatcttcacacccacaagtcagagaagctgtagtcatagcgcgggatgataacgctcaagaaaaacaaatcatcgcttatattacctataactccatcaaacctcagcttgataatctgcgtgatttcctaaaagcaaggctacctgattttatgattccagccgcttttgtgatgctggagcatcttcctttaactcccagtggtaaagtagaccgtaaggcattacctaagcctgatttatttaattatagtgaacataattcctatgtagcgcctcggaatgaagttgaagaaaaattagtacaaatctggtcgaatattctgcatttacctaaagtaggtgtgacagaaaactttttcgctattggtggtaattccctcaaagctctacatttaatttctcaaattgaagagttatttgctaaagagatatccttagcaacacttttaacaaatccagtaattgcagatttagccaaggttattcaagcaaacaaccaaatccataattcacccctagttccaattcaaccacaaggtaagcagcagcctttcttttgtatacatcctgctggtggtcatgttttatgctattttaaactcgcacaatatataggaactgaccaaccattttatggcttacaagctcaaggattttatggagatgaagcacccttgacgcgagttgaagatatggctagtctctacgtcaaaactattagagaatttcaaccccaagggccttatcgtgtcggggggtggtcatttggtggagtcgtagcttatgaagtagcacagcagttacatagacaaggacaagaagtatctttactagcaatattagattcttacgtaccgattctgctggataaacaaaaacccattgatgacgtttatttagttggtgttctctccagagtttttggcggtatgtttggtcaagataatctagtcacacctgaagaaatagaaaatttaactgtagaagaaaaaattaattacatcattgataaagcacggagcgctagaatattcccgcctggtgtagaacgtcaaaataatcgccgtattcttgatgttttggtgggaactttaaaagcaacttattcctatataagacaaccatatccaggaaaagtcactgtatttcgagccagggaaaaacatattatggctcctgacccgaccttagtttgggtagaattattttctgtaatggcggctcaagaaattaagattattgatgtccctggaaaccattattcgtttgttctagaaccccatgtacaggttttagcacagcgtttacaagattgtctggaaaataattcataa

SEQ ID NO: 18 - DNA, 2379 bp, Escherichia coli W3110
atgtccaacaatggctcgtcaccgctggtgctttggtataaccaactcggcatgaatgatgtagacagggttgggggcaaaaatgcctccctgggtgaaatgattactaatctttccggaatgggtgtttccgttccgaatggtttcgccacaaccgccgacgcgtttaaccagtttctggaccaaagcggcgtaaaccagcgcatttatgaactgctggataaaacggatattgacgatgttactcagcttgcgaaagcgggcgcgcaaatccgccagtggattatcgacactcccttccagcctgagctggaaaacgccatccgcgaagcctatgcacagctttccgccgatgacgaaaacgcctcttttgcggtgcgctcctccgccaccgcagaagatatgccggacgcttcttttgccggtcagcaggaaaccttcctcaacgttcagggttttgacgccgttctcgtggcagtgaaacatgtatttgcttctctgtttaacgatcgcgccatctcttatcgtgtgcaccagggttacgatcaccgtggtgtggcgctctccgccggtgttcaacggatggtgcgctctgacctcgcatcatctggcgtgatgttctccattgataccgaatccggctttgaccaggtggtgtttatcacttccgcatggggccttggtgagatggtcgtgcagggtgcggttaacccggatgagttttacgtgcataaaccgacactggcggcgaatcgcccggctatcgtgcgccgcaccatggggtcgaaaaaaatccgcatggtttacgcgccgacccaggagcacggcaagcaggttaaaatcgaagacgtaccgcaggaacagcgtgacatcttctcgctgaccaacgaagaagtgcaggaactggcaaaacaggccgtacaaattgagaaacactacggtcgcccgatggatattgagtgggcgaaagatggccacaccggtaaactgttcattgtgcaggcgcgtccggaaaccgtgcgctcacgcggtcaggtcatggagcgttatacgctgcattcacagggtaagattatcgccgaaggccgtgctatcggtcatcgcatcggtgcgggtccggtgaaagtcatccatgacatcagcgaaatgaaccgcatcgaacctggcgacgtgctggttactgacatgaccgacccggactgggaaccgatcatgaagaaagcatctgccatcgtcaccaaccgtggcggtcgtacctgtcacgcggcgatcatcgctcgtgaactgggcattccggcggtagtgggctgtggagatgcaacagaacggatgaaagacggtgagaacgtcactgtttcttgtgccgaaggtgataccggttacgtctatgcggagttgctggaatttagcgtgaaaagctccagcgtagaaacgatgccggatctgccgttgaaagtgatgatgaacgtcggtaacccggaccgtgctttcgacttcgcctgcctaccgaacgaaggcgtgggccttgcgcgtctggaatttatcatcaaccgtatgattggcgtccacccacgcgcactgcttgagtttgacgatcaggaaccgcagttgcaaaacgaaatccgcgagatgatgaaaggttttgattctccgcgtgaattttacgttggtcgtctgactgaagggatcgcgacgctgggtgccgcgttttatccgaagcgcgtcattgtccgtctctctgattttaaatcgaacgaatatgccaacctggtcggtggtgagcgttacgagccagatgaagagaacccgatgctcggcttccgtggcgcgggccgctatgtttccgacagcttccgcgactgtttcgcgctggagtgtgaagcagtgaaacgtgtgcgcaacgacatgggactgaccaacgttgagatcatgatcccgttcgtgcgtaccgtagatcaggcgaaagcggtggttgaagaactggcgcgtcaggggctgaaacgtggcgagaacgggctgaaaatcatcatgatgtgtgaaatcccgtccaacgccttgctggccgagcagttcctcgaatatttcgacggcttctcaattggctcaaacgatatgacgcagctggcgctcggtctggaccgtgactccggcgtggtgtctgaattgttcgatgagcgcaacgatgcggtgaaagcactgctgtcgatggctatccgtgccgcgaagaaacagggcaaatatgtcgggatttgcggtcagggtccgtccgaccacgaagactttgccgcatggttgatggaagaggggatcgatagcctgtctctgaacccggacaccgtggtgcaaacctggttaagcctggctgaactgaagaaataa

SEQ ID NO: 19 - protein, 792 aa, Escherichia coli W3110
MSNNGSSPLVLWYNQLGMNDVDRVGGKNASLGEMITNLSGMGVSVPNGFATTADAFNQFLDQSGVNQRIYELLDKTDIDDVTQLAKAGAQIRQWIIDTPFQPELENAIREAYAQLSADDENASFAVRSSATAEDMPDASFAGQQETFLNVQGFDAVLVAVKHVFASLFNDRAISYRVHQGYDHRGVALSAGVQRMVRSDLASSGVMFSIDTESGFDQVVFITSAWGLGEMVVQGAVNPDEFYVHKPTLAANRPAIVRRTMGSKKIRMVYAPTQEHGKQVKIEDVPQEQRDIFSLTNEEVQELAKQAVQIEKHYGRPMDIEWAKDGHTGKLFIVQARPETVRSRGQVMERYTLHSQGKIIAEGRAIGHRIGAGPVKVIHDISEMNRIEPGDVLVTDMTDPDWEPIMKKASAIVTNRGGRTCHAAIIARELGIPAVVGCGDATERMKDGENVTVSCAEGDTGYVYAELLEFSVKSSSVETMPDLPLKVMMNVGNPDRAFDFACLPNEGVGLARLEFIINRMIGVHPRALLEFDDQEPQLQNEIREMMKGFDSPREFYVGRLTEGIATLGAAFYPKRVIVRLSDFKSNEYANLVGGERYEPDEENPMLGFRGAGRYVSDSFRDCFALECEAVKRVRNDMGLTNVEIMIPFVRTVDQAKAVVEELARQGLKRGENGLKIIMMCEIPSNALLAEQFLEYFDGFSIGSNDMTQLALGLDRDSGVVSELFDERNDAVKALLSMAIRAAKKQGKYVGICGQGPSDHEDFAAWLMEEGIDSLSLNPDTVVQTWLSLAELKK

SEQ ID NO: 20 - DNA, 34 bp, Artificial Sequence
  Pn-pps_F
aaaggagctcactagttttgttcttcccgtgatg

SEQ ID NO: 21 - DNA, 36 bp, Artificial Sequence
  Pn-pps_R
cagggtgtggtcgatatcttatttcttcagttcagc

SEQ ID NO: 22 - DNA, 32 bp, Artificial Sequence
  pps_F
taggagattaacatatgtccaacaatggctcg

SEQ ID NO: 23 - DNA, 1992 bp, Escherichia coli W3110
atgtcctcacgtaaagagcttgccaatgctattcgtgcgctgagcatggacgcagtacagaaagccaaatccggtcacccgggtgcccctatgggtatggctgacattgccgaagtcctgtggcgtgatttcctgaaacacaacccgcagaatccgtcctgggctgaccgtgaccgcttcgtgctgtccaacggccacggctccatgctgatctacagcctgctgcacctcaccggttacgatctgccgatggaagaactgaaaaacttccgtcagctgcactctaaaactccgggtcacccggaagtgggttacaccgctggtgtggaaaccaccaccggtccgctgggtcagggtattgccaacgcagtcggtatggcgattgcagaaaaaacgctggcggcgcagtttaaccgtccgggccacgacattgtcgaccactacacctacgccttcatgggcgacggctgcatgatggaaggcatctcccacgaagtttgctctctggcgggtacgctgaagctgggtaaactgattgcattctacgatgacaacggtatttctatcgatggtcacgttgaaggctggttcaccgacgacaccgcaatgcgtttcgaagcttacggctggcacgttattcgcgacatcgacggtcatgacgcggcatctatcaaacgcgcagtagaagaagcgcgcgcagtgactgacaaaccttccctgctgatgtgcaaaaccatcatcggtttcggttccccgaacaaagccggtacccacgactcccacggtgcgccgctgggcgacgctgaaattgccctgacccgcgaacaactgggctggaaatatgcgccgttcgaaatcccgtctgaaatctatgctcagtgggatgcgaaagaagcaggccaggcgaaagaatccgcatggaacgagaaattcgctgcttacgcgaaagcttatccgcaggaagccgctgaatttacccgccgtatgaaaggcgaaatgccgtctgacttcgacgctaaagcgaaagagttcatcgctaaactgcaggctaatccggcgaaaatcgccagccgtaaagcgtctcagaatgctatcgaagcgttcggtccgctgttgccggaattcctcggcggttctgctgacctggcgccgtctaacctgaccctgtggtctggttctaaagcaatcaacgaagatgctgcgggtaactacatccactacggtgttcgcgagttcggtatgaccgcgattgctaacggtatctccctgcacggtggcttcctgccgtacacctccaccttcctgatgttcgtggaatacgcacgtaacgccgtacgtatggctgcgctgatgaaacagcgtcaggtgatggtttacacccacgactccatcggtctgggcgaagacggcccgactcaccagccggttgagcaggtcgcttctctgcgcgtaaccccgaacatgtctacatggcgtccgtgtgaccaggttgaatccgcggtcgcgtggaaatacggtgttgagcgtcaggacggcccgaccgcactgatcctctcccgtcagaacctggcgcagcaggaacgaactgaagagcaactggcaaacatcgcgcgcggtggttatgtgctgaaagactgcgccggtcagccggaactgattttcatcgctaccggttcagaagttgaactggctgttgctgcctacgaaaaactgactgccgaaggcgtgaaagcgcgcgtggtgtccatgccgtctaccgacgcatttgacaagcaggatgctgcttaccgtgaatccgtactgccgaaagcggttactgcacgcgttgctgtagaagcgggtattgctgactactggtacaagtatgttggcctgaacggtgctatcgtcggtatgaccaccttcggtgaatctgctccggcagagctgctgtttgaagagttcggcttcactgttgataacgttgttgcgaaagcaaaagaactgctgtaa

SEQ ID NO: 24 - protein, 663 aa, Escherichia coli W3110
MSSRKELANAIRALSMDAVQKAKSGHPGAPMGMADIAEVLWRDFLKHNPQNPSWADRDRFVLSNGHGSMLIYSLLHLTGYDLPMEELKNFRQLHSKTPGHPEVGYTAGVETTTGPLGQGIANAVGMAIAEKTLAAQFNRPGHDIVDHYTYAFMGDGCMMEGISHEVCSLAGTLKLGKLIAFYDDNGISIDGHVEGWFTDDTAMRFEAYGWHVIRDIDGHDAASIKRAVEEARAVTDKPSLLMCKTIIGFGSPNKAGTHDSHGAPLGDAEIALTREQLGWKYAPFEIPSEIYAQWDAKEAGQAKESAWNEKFAAYAKAYPQEAAEFTRRMKGEMPSDFDAKAKEFIAKLQANPAKIASRKASQNAIEAFGPLLPEFLGGSADLAPSNLTLWSGSKAINEDAAGNYIHYGVREFGMTAIANGISLHGGFLPYTSTFLMFVEYARNAVRMAALMKQRQVMVYTHDSIGLGEDGPTHQPVEQVASLRVTPNMSTWRPCDQVESAVAWKYGVERQDGPTALILSRQNLAQQERTEEQLANIARGGYVLKDCAGQPELIFIATGSEVELAVAAYEKLTAEGVKARVVSMPSTDAFDKQDAAYRESVLPKAVTARVAVEAGIADYWYKYVGLNGAIVGMTTFGESAPAELLFEEFGFTVDNVVAKAKELL

SEQ ID NO: 25 - DNA, 34 bp, Artificial Sequence
  Pn-tktA_F
aaaggagctcactagtgttttctcttccgcacaa

SEQ ID NO: 26 - DNA, 36 bp, Artificial Sequence
  Pn-tktA_R
cagggtgtggtcgatatcttacagcagttcttttgc

SEQ ID NO: 27 - DNA, 32 bp, Artificial Sequence
  tktA_F
taggagattaacatatgtcctcacgtaaagag

SEQ ID NO: 28 - DNA, 26 bp, Artificial Sequence
  Pcj1-aroG_R
cccgcggtttacccgcgacgcgcttt

SEQ ID NO: 29 - DNA, 26 bp, Artificial Sequence
  Pcj1-pps_F
gcgggtaaaccgcgggcttattccat

SEQ ID NO: 30 - DNA, 26 bp, Artificial Sequence
  Pcj1-pps_R
cccgcggtttatttcttcagttcagc

SEQ ID NO: 31 - DNA, 26 bp, Artificial Sequence
  Pcj1-tktA_F
agaaataaaccgcgggcttattccat

SEQ ID NO: 32 - DNA, 71 bp, Artificial Sequence
  aroD deletion cassette_F
tcatggggttcggtgcctgacaggctgaccgcgtgcagaaagggtaaaaagctggagctgcttcgaagttc

SEQ ID NO: 33 - DNA, 79 bp, Artificial Sequence
  aroD deletion cassette_R
atatattttttagttcggcggggagggtgttcccgccgaaatattattgcgccatggtccatatgaatatcctccttag

SEQ ID NO: 34 - DNA, 23 bp, Artificial Sequence
  aroD deletion_F
caaagatttccctctggaatatg

SEQ ID NO: 35 - DNA, 21 bp, Artificial Sequence
  aroD deletion_R
cagatgtgattttccctacgc

SEQ ID NO: 36 - DNA, 1389 bp, Corynebacterium glutamicum ATCC13032
gtgagttggacagttgatatccctaaagaagttctccctgatttgccaccattgccagaaggcatgcagcagcagttcgaggacaccatttcccgtgacgctaagcagcaacctacgtgggatcgtgcacaggcagaaaacgtgcgcaagatccttgagtcggttcctccaatcgttgttgcccctgaggtacttgagctgaagcagaagcttgctgatgttgccaacggtaaggccttcctcttgcagggtggtgactgtgcggaaactttcgagtcaaacactgagccgcacattcgcgccaacgtaaagactctgctgcagatggcagttgttttgacctacggtgcatccactcctgtgatcaagatggctcgtattgctggtcagtacgcaaagcctcgctcttctgatctggatggaaatggtctgccaaactaccgtggcgatatcgtcaacggtgtggaggcaaccccagaggctcgtcgccacgatcctgcccgcatgatccgtgcttacgctaacgcttctgctgcgatgaacttggtgcgcgcgctcaccagctctggcaccgctgatctttaccgtctcagcgagtggaaccgcgagttcgttgcgaactccccagctggtgcacgctacgaggctcttgctcgtgagatcgactccggtctgcgcttcatggaagcatgtggcgtgtccgatgagtccctgcgtgctgcagatatctactgctcccacgaggctttgctggtggattacgagcgttccatgctgcgtcttgcaaccgatgaggaaggcaacgaggaactttacgatctttcagctcaccagctgtggatcggcgagcgcacccgtggcatggatgatttccatgtgaacttcgcatccatgatctctaacccaatcggcatcaagattggtcctggtatcacccctgaagaggctgttgcatacgctgacaagctcgatccgaacttcgagcctggccgtttgaccatcgttgctcgcatgggccacgacaaggttcgctccgtacttcctggtgttatccaggctgttgaggcatccggacacaaggttatttggcagtccgatccgatgcacggcaacactttcaccgcatccaatggctacaagacccgtcacttcgacaaggttatcgatgaggtccagggcttcttcgaggtccaccgcgcattgggcacccacccaggcggaatccacattgagttcactggtgaagatgtcaccgagtgcctcggtggcgctgaagacatcaccgatgttgatctgccaggccgctacgagtccgcatgcgatcctcgcctgaacactcagcagtctttggagttggctttcctcgttgcagaaatgctgcgtaactaa

SEQ ID NO: 37 - protein, 462 aa, Corynebacterium glutamicum ATCC13032
MSWTVDIPKEVLPDLPPLPEGMQQQFEDTISRDAKQQPTWDRAQAENVRKILESVPPIVVAPEVLELKQKLADVANGKAFLLQGGDCAETFESNTEPHIRANVKTLLQMAVVLTYGASTPVIKMARIAGQYAKPRSSDLDGNGLPNYRGDIVNGVEATPEARRHDPARMIRAYANASAAMNLVRALTSSGTADLYRLSEWNREFVANSPAGARYEALAREIDSGLRFMEACGVSDESLRAADIYCSHEALLVDYERSMLRLATDEEGNEELYDLSAHQLWIGERTRGMDDFHVNFASMISNPIGIKIGPGITPEEAVAYADKLDPNFEPGRLTIVARMGHDKVRSVLPGVIQAVEASGHKVIWQSDPMHGNTFTASNGYKTRHFDKVIDEVQGFFEVHRALGTHPGGIHIEFTGEDVTECLGGAEDITDVDLPGRYESACDPRLNTQQSLELAFLVAEMLRN

SEQ ID NO: 38 - DNA, 36 bp, Artificial Sequence
  Pn-cgl aroG_F
ggtatcgataagcttgattcgatctgctcccattcc

SEQ ID NO: 39 - DNA, 36 bp, Artificial Sequence
  Pn-cgl aroG_R
cggtggcggccgctctagttagttacgcagcatttc

SEQ ID NO: 40 - DNA, 39 bp, Artificial Sequence
  Pcj7-cgl aroG_F
cgaaaggaaacactcgatgtgagttggacagttgatatc

SEQ ID NO: 41 - DNA, 37 bp, Artificial Sequence
  Pcj7-cgl aroG_R
ccgccaaaacagctctagtttagttacgcagcatttc

SEQ ID NO: 42 - DNA, 60 bp, Artificial Sequence
  Ava_ABCD_F
ccaaacaccaacaaaaggctctacccatatgatgagtatcgtccaagcaaagtttgaagc

SEQ ID NO: 43 - DNA, 60 bp, Artificial Sequence
  Ava_ABCD_R
agagggtgacgctagtcagaactagtttatgaattattttccagacaatcttgtaaacgc

SEQ ID NO: 44 - DNA, 54 bp, Artificial Sequence
  O2 promoter_F
ccaaacaccaacaaaaggctctacccatatgcaataatcgtgaattttggcagc

SEQ ID NO: 45 - DNA, 53 bp, Artificial Sequence
  O2 promoter_R
caaactttgcttggacgatactcattgttttgatctcctccaataatctatgc

SEQ ID NO: 46 - DNA, 37 bp, Artificial Sequence
  Pn-cgl tkt_F
ggtatcgataagcttgataagattgatcacacctttg

SEQ ID NO: 47 - DNA, 36 bp, Artificial Sequence
  Pn-cgl tkt_R
cggtggcggccgctctagttaaccgttaatggagtc

SEQ ID NO: 48 - DNA, 36 bp, Artificial Sequence
  Pcj7-cgl tkt_F
cgaaaggaaacactcgatttgaccaccttgacgctg

SEQ ID NO: 49 - DNA, 37 bp, Artificial Sequence
  Pcj7-cgl tkt_R
ccgccaaaacagctctagtttaaccgttaatggagtc

SEQ ID NO: 50 - DNA, 36 bp, Artificial Sequence
  Ptrc-cgl pps_F
tcacacaggaaagatatcatgaccaacagtttgaac

SEQ ID NO: 51 - DNA, 39 bp, Artificial Sequence
  Ptrc-cgl pps_R
ccgccaaaacagctctagtttacttcgtgccggtcattg

SEQ ID NO: 52 - DNA, 36 bp, Artificial Sequence
  Pcj7-cgl pps_F
cgaaaggaaacactcgatatgaccaacagtttgaac

SEQ ID NO: 53 - DNA, 37 bp, Artificial Sequence
  Pcj7-aroG_F
ttagaaatggaacctaaaagaaacatcccagcgctac

SEQ ID NO: 54 - DNA, 36 bp, Artificial Sequence
  Pcj7-aroG_R
tagcgctgggatgtttctttagttacgcagcatttc

SEQ ID NO: 55 - DNA, 36 bp, Artificial Sequence
  Pcj7-tkt_F
gaaatgctgcgtaactaaagaaacatcccagcgcta

SEQ ID NO: 56 - DNA, 37 bp, Artificial Sequence
  Pcj7-tkt_R
gtagcgctgggatgtttctttaaccgttaatggagtc

SEQ ID NO: 57 - DNA, 37 bp, Artificial Sequence
  Pcj7-pps_F
gactccattaacggttaaagaaacatcccagcgctac

SEQ ID NO: 58 - DNA, 39 bp, Artificial Sequence
  Pcj7-pps_R
ggagaatactgtcgttcatttacttcgtgccggtcattg

SEQ ID NO: 59 - DNA, 26 bp, Artificial Sequence
  pGPD_F
ggatccacagtttattcctggcatcc

SEQ ID NO: 60 - DNA, 40 bp, Artificial Sequence
  pGPD_R
tttgcttggacgatactcatttgtttgtttatgtgtgttt

SEQ ID NO: 61 - DNA, 40 bp, Artificial Sequence
  Ava_A_F
aaacacacataaacaaacaaatgagtatcgtccaagcaaa

SEQ ID NO: 62 - DNA, 40 bp, Artificial Sequence
  Ava_A_R
ggatgccaggaataaactgtttatttaacactcccgatta

SEQ ID NO: 63 - DNA, 40 bp, Artificial Sequence
  pGPD_F
taatcgggagtgttaaataaacagtttattcctggcatcc

SEQ ID NO: 64 - DNA, 40 bp, Artificial Sequence
  pGPD_R
tggacaatcacatttgtcaattgtttgtttatgtgtgttt

SEQ ID NO: 65 - DNA, 40 bp, Artificial Sequence
  Ava_B_F
aaacacacataaacaaacaattgacaaatgtgattgtcca

SEQ ID NO: 66 - DNA, 26 bp, Artificial Sequence
  Ava_B_R
gtcgacttaaggttgtattctgcgga

SEQ ID NO: 67 - DNA, 26 bp, Artificial Sequence
  pGPD_F
ggatccacagtttattcctggcatcc

SEQ ID NO: 68 - DNA, 40 bp, Artificial Sequence
  pGPD_R
aaagagattgattgtgccatttgtttgtttatgtgtgttt

SEQ ID NO: 69 - DNA, 40 bp, Artificial Sequence
  Ava_C_F
aaacacacataaacaaacaaatggcacaatcaatctcttt

SEQ ID NO: 70 - DNA, 40 bp, Artificial Sequence
  Ava_C_R
ggatgccaggaataaactgtttagtcgccccctaattcca

SEQ ID NO: 71 - DNA, 40 bp, Artificial Sequence
  pGPD_F
tggaattagggggcgactaaacagtttattcctggcatcc

SEQ ID NO: 72 - DNA, 40 bp, Artificial Sequence
  pGPD_R
aggatattaagtactggcatttgtttgtttatgtgtgttt

SEQ ID NO: 73 - DNA, 40 bp, Artificial Sequence
  Ava_D_F
aaacacacataaacaaacaaatgccagtacttaatatcct

SEQ ID NO: 74 - DNA, 26 bp, Artificial Sequence
  Ava_D_R
gtcgactcaattttgtaacacctttt

SEQ ID NO: 75 - DNA, 26 bp, Artificial Sequence
  TKL1_F
ggatccatgactcaattcactgacat

SEQ ID NO: 76 - DNA, 26 bp, Artificial Sequence
  TKL1_R
gtcgacttagaaagcttttttcaaag

SEQ ID NO: 77 - DNA, 26 bp, Artificial Sequence
  ARO4_F
ggatccatgagtgaatctccaatgtt

SEQ ID NO: 78 - DNA, 26 bp, Artificial Sequence
  ARO4_R
gtcgacctatttcttgttaacttctc

SEQ ID NO: 79 - DNA, 26 bp, Artificial Sequence
  pps_F
ggatccatgtccaacaatggctcgtc

SEQ ID NO: 80 - DNA, 26 bp, Artificial Sequence
  pps_R
gtcgacttatttcttcagttcagcca

SEQ ID NO: 81 - DNA, 26 bp, Artificial Sequence
  pGPD_F
ggatccacagtttattcctggcatcc

SEQ ID NO: 82 - DNA, 40 bp, Artificial Sequence
  pGPD_R
atgtcagtgaattgagtcatttgtttgtttatgtgtgttt

SEQ ID NO: 83 - DNA, 40 bp, Artificial Sequence
  TKL1_F
aaacacacataaacaaacaaatgactcaattcactgacat

SEQ ID NO: 84 - DNA, 40 bp, Artificial Sequence
  TKL1_R
ggatgccaggaataaactgtttagaaagcttttttcaaag

SEQ ID NO: 85 - DNA, 40 bp, Artificial Sequence
  pGPD_F
ctttgaaaaaagctttctaaacagtttattcctggcatcc

SEQ ID NO: 86 - DNA, 40 bp, Artificial Sequence
  pGPD_R
aacattggagattcactcatttgtttgtttatgtgtgttt

SEQ ID NO: 87 - DNA, 40 bp, Artificial Sequence
  ARO4_F
aaacacacataaacaaacaaatgagtgaatctccaatgtt

SEQ ID NO: 88 - DNA, 26 bp, Artificial Sequence
  ARO4_R
gtcgacctatttcttgttaacttctc

SEQ ID NO: 89 - DNA, 438 bp, Corynebacterium glutamicum ATCC13032
atgcctggaaaaattctcctcctcaacggcccaaacctgaacatgctgggcaaacgcgagcctgacatttacggacacgacaccttggaagacgtcgtcgcgctggcaaccgctgaggctgcgaagcacggccttgaggttgaggcgctgcagagcaatcacgaaggtgagctaatcgatgcgctgcacaacgctcgcggcacccacatcggttgcgtgattaaccccggcggcctgactcacacttcggtggcgcttttggatgctgtgaaggcgtctgagcttcctaccgttgaggtgcacatttccaatccgcatgcccgtgaagagttccgccaccattcttacatttccctcgccgcggtctccgttatcgctggcgctggcatccagggttaccgtttcgcggtcgatatcctggcaaatctcaaaaagtag

SEQ ID NO: 90 - protein, 145 aa, Corynebacterium glutamicum ATCC13032
MPGKILLLNGPNLNMLGKREPDIYGHDTLEDVVALATAEAAKHGLEVEALQSNHEGELIDALHNARGTHIGCVINPGGLTHTSVALLDAVKASELPTVEVHISNPHAREEFRHHSYISLAAVSVIAGAGIQGYRFAVDILANLKK

SEQ ID NO: 91 - DNA, 32 bp, Artificial Sequence
  aroD_F
gatcggatcccactggacgtttgggtgagacc

SEQ ID NO: 92 - DNA, 37 bp, Artificial Sequence
  aroD_R
gatcggatccactagttttaggttccatttctaattg

SEQ ID NO: 93 - DNA, 29 bp, Artificial Sequence
  aroD_F
gatcactagtatgaacgacagtattctcc

SEQ ID NO: 94 - DNA, 30 bp, Artificial Sequence
  aroD_D
gatcaagcttgttacatcctgacgttgtgg

SEQ ID NO: 95 - DNA, 2103 bp, Corynebacterium glutamicum ATCC13032
ttgaccaccttgacgctgtcacctgaacttcaggcgctcactgtacgcaattacccctctgattggtccgatgtggacaccaaggctgtagacactgttcgtgtcctcgctgcagacgctgtagaaaactgtggctccggccacccaggcaccgcaatgagcctggctccccttgcatacaccttgtaccagcgggttatgaacgtagatccacaggacaccaactgggcaggccgtgaccgcttcgttctttcttgtggccactcctctttgacccagtacatccagctttacttgggtggattcggccttgagatggatgacctgaaggctctgcgcacctgggattccttgaccccaggacaccctgagtaccgccacaccaagggcgttgagatcaccactggccctcttggccagggtcttgcatctgcagttggtatggccatggctgctcgtcgtgagcgtggcctattcgacccaaccgctgctgagggcgaatccccattcgaccaccacatctacgtcattgcttctgatggtgacctgcaggaaggtgtcacctctgaggcatcctccatcgctggcacccagcagctgggcaacctcatcgtgttctgggatgacaaccgcatctccatcgaagacaacactgagatcgctttcaacgaggacgttgttgctcgttacaaggcttacggctggcagaccattgaggttgaggctggcgaggacgttgcagcaatcgaagctgcagtggctgaggctaagaaggacaccaagcgacctaccttcatccgcgttcgcaccatcatcggcttcccagctccaactatgatgaacaccggtgctgtgcacggtgctgctcttggcgcagctgaggttgcagcaaccaagactgagcttggattcgatcctgaggctcacttcgcgatcgacgatgaggttatcgctcacacccgctccctcgcagagcgcgctgcacagaagaaggctgcatggcaggtcaagttcgatgagtgggcagctgccaaccctgagaacaaggctctgttcgatcgcctgaactcccgtgagcttccagcgggctacgctgacgagctcccaacatgggatgcagatgagaagggcgtcgcaactcgtaaggcttccgaggctgcacttcaggcactgggcaagacccttcctgagctgtggggcggttccgctgacctcgcaggttccaacaacaccgtgatcaagggctccccttccttcggccctgagtccatctccaccgagacctggtctgctgagccttacggccgtaacctgcacttcggtatccgtgagcacgctatgggatccatcctcaacggcatttccctccacggtggcacccgcccatacggcggaaccttcctcatcttctccgactacatgcgtcctgcagttcgtcttgcagctctcatggagaccgacgcttactacgtctggacccacgactccatcggtctgggcgaagatggcccaacccaccagcctgttgaaaccttggctgcactgcgcgccatcccaggtctgtccgtcctgcgtcctgcagatgcgaacgagaccgcccaggcttgggctgcagcacttgagtacaaggaaggccctaagggtcttgcactgacccgccagaacgttcctgttctggaaggcaccaaggagaaggctgctgaaggcgttcgccgcggtggctacgtcctggttgagggttccaaggaaaccccagatgtgatcctcatgggctccggctccgaggttcagcttgcagttaacgctgcgaaggctctggaagctgagggcgttgcagctcgcgttgtttccgttccttgcatggattggttccaggagcaggacgcagagtacatcgagtccgttctgcctgcagctgtgaccgctcgtgtgtctgttgaagctggcatcgcaatgccttggtaccgcttcttgggcacccagggccgtgctgtctcccttgagcacttcggtgcttctgcggattaccagaccctgtttgagaagttcggcatcaccaccgatgcagtcgtggcagcggccaaggactccattaacggttaa

SEQ ID NO: 96 - protein, 700 aa, Corynebacterium glutamicum ATCC13032
MTTLTLSPELQALTVRNYPSDWSDVDTKAVDTVRVLAADAVENCGSGHPGTAMSLAPLAYTLYQRVMNVDPQDTNWAGRDRFVLSCGHSSLTQYIQLYLGGFGLEMDDLKALRTWDSLTPGHPEYRHTKGVEITTGPLGQGLASAVGMAMAARRERGLFDPTAAEGESPFDHHIYVIASDGDLQEGVTSEASSIAGTQQLGNLIVFWDDNRISIEDNTEIAFNEDVVARYKAYGWQTIEVEAGEDVAAIEAAVAEAKKDTKRPTFIRVRTIIGFPAPTMMNTGAVHGAALGAAEVAATKTELGFDPEAHFAIDDEVIAHTRSLAERAAQKKAAWQVKFDEWAAANPENKALFDRLNSRELPAGYADELPTWDADEKGVATRKASEAALQALGKTLPELWGGSADLAGSNNTVIKGSPSFGPESISTETWSAEPYGRNLHFGIREHAMGSILNGISLHGGTRPYGGTFLIFSDYMRPAVRLAALMETDAYYVWTHDSIGLGEDGPTHQPVETLAALRAIPGLSVLRPADANETAQAWAAALEYKEGPKGLALTRQNVPVLEGTKEKAAEGVRRGGYVLVEGSKETPDVILMGSGSEVQLAVNAAKALEAEGVAARVVSVPCMDWFQEQDAEYIESVLPAAVTARVSVEAGIAMPWYRFLGTQGRAVSLEHFGASADYQTLFEKFGITTDAVVAAAKDSING

SEQ ID NO: 97 - DNA, 1095 bp, Corynebacterium glutamicum ATCC13032
atgaccaacagtttgaacatcccgtttgtccagcgcttcgatgaaggcctggatcctgttctagaagtactcggtggcaagggcgcttcactagtcaccatgacagatgctggaatgcccgttccacctggatttgtggtcactactgccagctttgatgaattcatccgtgaagcaggggttgctgaacacatcgataaattcctaaacgatctcgatgcagaagatgttaaggaagtggatcgagtttctgcgatcatccgcgatgagctgtgcagtcttgacgttccagagaatgctcgtttcgcagtgcaccaggcttatcgcgatctcatggaacgatgcggtggcgacgtcccggttgctgtccggtcatcggccactgccgaagatctgcccgatgcttccttcgcagggcaacaggacacctatctgtggcaagtcggtttgagcgctgtcactgaacacatccgtaaatgctgggcttcgctgttcacttcccgtgccattatctaccgtctgaaaaacaacatccccaatgagggcctctccatggcggtagttgttcaaaaaatggtcaactctcgtgtcgcaggcgtggcaatcactatgaatccttccaacggcgaccgctcgaagatcaccatcgattcctcatggggtgttggtgaaatggtggtctcaggtgaagtgacaccagacaatatcttgctggacaagatcacgctgcaggttgtctccgaacacattggaagcaaacacgctgaactcatccccgatgccaccagtggaagcctcgtggaaaagcccgttgatgaagaacgcgcaaaccgccgcagtctgactgatgaggaaatgctcgctgtggcacaaatggctaagcgtgcagaaaaacactacaagtgcccacaagatatcgaatgggcgctggacgctgatctgccagatggagaaaaccttctgttattgcaatcccgcccggaaactatccactccaacggtgtgaagaaggaaaccccaactccgcaggctgccaaaaccataggcaccttcgatttcagctcaatcaccgtcgcaatgaccggcacgaagtaa

SEQ ID NO: 98 - protein, 364 aa, Corynebacterium glutamicum ATCC13032
MTNSLNIPFVQRFDEGLDPVLEVLGGKGASLVTMTDAGMPVPPGFVVTTASFDEFIREAGVAEHIDKFLNDLDAEDVKEVDRVSAIIRDELCSLDVPENARFAVHQAYRDLMERCGGDVPVAVRSSATAEDLPDASFAGQQDTYLWQVGLSAVTEHIRKCWASLFTSRAIIYRLKNNIPNEGLSMAVVVQKMVNSRVAGVAITMNPSNGDRSKITIDSSWGVGEMVVSGEVTPDNILLDKITLQVVSEHIGSKHAELIPDATSGSLVEKPVDEERANRRSLTDEEMLAVAQMAKRAEKHYKCPQDIEWALDADLPDGENLLLLQSRPETIHSNGVKKETPTPQAAKTIGTFDFSSITVAMTGTK

SEQ ID NO: 99 - DNA, 3926 bp, Saccharomyces cerevisiae
ttggtcatgcttatgaagctatactaaccccacaagcattacatggtgaatgtgtgtccattggtatggttaaagaggcggaattatcccgttatttcggtattctctcccctacccaagttgcacgtctatccaagattttggttgcctacgggttgcctgtttcgcctgatgagaaatggtttaaagagctaaccttacataagaaaacaccattggatatcttattgaagaaaatgagtattgacaagaaaaacgagggttccaaaaagaaggtggtcattttagaaagtattggtaagtgctatggtgactccgctcaatttgttagcgatgaagacctgagatttattctaacagatgaaaccctcgtttaccccttcaaggacatccctgctgatcaacagaaagttgttatcccccctggttctaagtccatctccaatcgtgctttaattcttgctgccctcggtgaaggtcaatgtaaaatcaagaacttattacattctgatgatactaaacatatgttaaccgctgttcatgaattgaaaggtgctacgatatcatgggaagataatggtgagacggtagtggtggaaggacatggtggttccacattgtcagcttgtgctgaccccttatatctaggtaatgcaggtactgcatctagatttttgacttccttggctgccttggtcaattctacttcaagccaaaagtatatcgttttaactggtaacgcaagaatgcaacaaagaccaattgctcctttggtcgattctttgcgtgctaatggtactaaaattgagtacttgaataatgaaggttccctgccaatcaaagtttatactgattcggtattcaaaggtggtagaattgaattagctgctacagtttcttctcagtacgtatcctctatcttgatgtgtgccccatacgctgaagaacctgtaactttggctcttgttggtggtaagccaatctctaaattgtacgtcgatatgacaataaaaatgatggaaaaattcggtatcaatgttgaaacttctactacagaaccttacacttattatattccaaagggacattatattaacccatcagaatacgtcattgaaagtgatgcctcaagtgctacatacccattggccttcgccgcaatgactggtactaccgtaacggttccaaacattggttttgagtcgttacaaggtgatgccagatttgcaagagatgtcttgaaacctatgggttgtaaaataactcaaacggcaacttcaactactgtttcgggtcctcctgtaggtactttaaagccattaaaacatgttgatatggagccaatgactgatgcgttcttaactgcatgtgttgttgccgctatttcgcacgacagtgatccaaattctgcaaatacaaccaccattgaaggtattgcaaaccagcgtgtcaaagagtgtaacagaattttggccatggctacagagctcgccaaatttggcgtcaaaactacagaattaccagatggtattcaagtccatggtttaaactcgataaaagatttgaaggttccttccgactcttctggacctgtcggtgtatgcacatatgatgatcatcgtgtggccatgagtttctcgcttcttgcaggaatggtaaattctcaaaatgaacgtgacgaagttgctaatcctgtaagaatacttgaaagacattgtactggtaaaacctggcctggctggtgggatgtgttacattccgaactaggtgccaaattagatggtgcagaacctttagagtgcacatccaaaaagaactcaaagaaaagcgttgtcattattggcatgagagcagctggcaaaactactataagtaaatggtgcgcatccgctctgggttacaaattagttgacctagacgagctgtttgagcaacagcataacaatcaaagtgttaaacaatttgttgtggagaacggttgggagaagttccgtgaggaagaaacaagaattttcaaggaagttattcaaaattacggcgatgatggatatgttttctcaacaggtggcggtattgttgaaagcgctgagtctagaaaagccttaaaagattttgcctcatcaggtggatacgttttacacttacatagggatattgaggagacaattgtctttttacaaagtgatccttcaagacctgcctatgtggaagaaattcgtgaagtttggaacagaagggaggggtggtataaagaatgctcaaatttctctttctttgctcctcattgctccgcagaagctgagttccaagctctaagaagatcgtttagtaagtacattgcaaccattacaggtgtcagagaaatagaaattccaagcggaagatctgcctttgtgtgtttaacctttgatgacttaactgaacaaactgagaatttgactccaatctgttatggttgtgaggctgtagaggtcagagtagaccatttggctaattactctgctgatttcgtgagtaaacagttatctatattgcgtaaagccactgacagtattcctatcatttttactgtgcgaaccatgaagcaaggtggcaactttcctgatgaagagttcaaaaccttgagagagctatacgatattgccttgaagaatggtgttgaattccttgacttagaactaactttacctactgatatccaatatgaggttattaacaaaaggggcaacaccaagatcattggttcccatcatgacttccaaggattatactcctgggacgacgctgaatgggaaaacagattcaatcaagcgttaactcttgatgtggatgttgtaaaatttgtgggtacggctgttaatttcgaagataatttgagactggaacactttagggatacacacaagaataagcctttaattgcagttaatatgacttctaaaggtagcatttctcgtgttttgaataatgttttaacacctgtgacatcagatttattgcctaactccgctgcccctggccaattgacagtagcacaaattaacaagatgtatacatctatgggaggtatcgagcctaaggaactgtttgttgttggaaagccaattggccactctagatcgccaattttacataacactggctatgaaattttaggtttacctcacaagttcgataaatttgaaactgaatccgcacaattggtgaaagaaaaacttttggacggaaacaagaactttggcggtgctgcagtcacaattcctctgaaattagatataatgcagtacatggatgaattgactgatgctgctaaagttattggtgctgtaaacacagttataccattgggtaacaagaagtttaagggtgataataccgactggttaggtatccgtaatgccttaattaacaatggcgttcccgaatatgttggtcataccgctggtttggttatcggtgcaggtggcacttctagagccgccctttacgccttgcacagtttaggttgcaaaaagatcttcataatcaacaggacaacttcgaaattgaagccattaatagagtcacttccatctgaattcaacattattggaatagagtccactaaatctatagaagagattaaggaacacgttggcgttgctgtcagctgtgtaccagccgacaaaccattagatgacgaacttttaagtaagctggagagattccttgtgaaaggtgcccatgctgcttttgtaccaaccttattggaagccgcatacaaaccaagcgttactcccgttatgacaatttcacaagacaaatatcaatggcacgttgtccctggatcacaaatgttagtacaccaaggtgtagctcagtttgaaaagtggacaggattcaagggccctttcaaggccatttttgatgccgttacgaaagagtag

SEQ ID NO: 100 - DNA, 2247 bp, Anabaena variabilis ATCC29413
atgagtatcgtccaagcaaagtttgaagctaaggaaacatcttttcatgtagaaggttacgaaaagattgagtatgatttggtgtatgtagatggtatttttgaaatccagaattctgcactagcagatgtatatcaaggttttggacgatgcttggcgattgtagatgctaacgtcagtcggttgtatggtaatcaaattcaggcatatttccagtattatggtatagaactgaggctatttcctattaccattactgaaccagataagactattcaaactttcgagagagttatagatgtctttgcagatttcaaattagtccgcaaagaaccagtattagtcgtgggtggcggtttaattacagatgttgtcggctttgcttgttctacatatcgtcgcagcagcaattacatccgcattcctactacattgattggattaattgatgccagtgtagcaattaaggtagcagttaatcatcgcaaactgaaaaaccgtttgggtgcttatcatgcttctcgcaaagtatttttagatttctccttgttgcgtactctccctacagaccaagtacgtaacgggatggcggaattggtaaaaatcgctgtagtagcgcatcaagaagtttttgaattgttggagaagtacggcgaagaattactacgtactcattttggcaatatagatgcaactccagagattaaagaaatagcccatcgtttgacttacaaagctatccataagatgttggaattggaagttcccaacctgcatgagttagacctagatagggtgattgcttacggtcacacttggagtcccaccttggaacttgcgcctcgtctacccatgttccacggacacgccgttaatgtagatatggctttctcggcaacgatcgccgcccgtagaggatatattacaattgcagaacgcgatcgtattttaggattaatgagtcgcgttggtctatccctcgaccatcccatgttggatatagatattttgtggcgtggtactgaatctatcacattaactcgtgatggtttgttaagagctgctatgccaaaacccattggtgattgtgtcttcgtcaatgacctgacaagagaagaattagcagccgcattagctgaccacaaagaactttgtaccagttatccccgtggtggtgaaggtgtggatgtgtatcccgtttatcaaaaagaattaatcgggagtgttaaataatgacttttttgaattcaaaatgcaaaatactccacggatacactgcgcgagcgcggtagcatttctgttcgcggagcgtcccgtagggaaagagaaggctacgcaaataatcggacactaattgtctttaattttgaattttgaattttgaattttgaattggagcgaagcgacttgacaaatgtgattgtccaaccaacagctagacctgttacaccattgggaattttaaccaagcagttagaagccatagtccaagaggttaagcaacatccagatttacctggggaattgatagcaaacatccatcaggcttggcgtttagccgcaggtatagacccttatttggaagaatgcaccactccagaatctcctgaactcgctgcattggcaaaaaccacagccaccgaagcctggggagaacacttccacggaggtacaaccgtccgtcctctagaacaagagatgctttctggtcatatcgaaggacaaaccttaaagatgtttgttcacatgaccaaagctaaaaaagtcttagaaattgggatgtttaccggttattcggcgctggcgatggcggaagcattaccagaggatggactgcttgtggcttgtgaagttgacccttacgcggcggaaattggacagaaagcctttcaacaatctccccacggtggaaagattcgtgtggaattggatgcagccttagcaactcttgataagttagcagaagctggggagtcttttgacttggtatttatcgacgcagataaaaaagagtatgtagcctattttcacaagttgctaggtagcagtttgttagcaccagatggctttatttgtgtagataacaccttattacaaggggaagtttatctaccagcagaggaacgtagcgtcaatggtgaagcgatcgcgcaatttaatcatacagtagctatagacccccgtgtagaacaggttttgttgccgttgcgagatggtttaacaattatccgcagaatacaaccttaa

SEQ ID NO: 101 - DNA, 3532 bp, Anabaena variabilis ATCC29413
tccagaattctgcactagcagatgtatatcaaggttttggacgatgcttggcgattgtagatgctaacgtcagtcggttgtatggtaatcaaattcaggcatatttccagtattatggtatagaactgaggctatttcctattaccattactgaaccagataagactattcaaactttcgagagagttatagatgtctttgcagatttcaaattagtccgcaaagaaccagtattagtcgtgggtggcggtttaattacagatgttgtcggctttgcttgttctacatatcgtcgcagcagcaattacatccgcattcctactacattgattggattaattgatgccagtgtagcaattaaggtagcagttaatcatcgcaaactgaaaaaccgtttgggtgcttatcatgcttctcgcaaagtatttttagatttctccttgttgcgtactctccctacagaccaagtacgtaacgggatggcggaattggtaaaaatcgctgtagtagcgcatcaagaagtttttgaattgttggagaagtacggcgaagaattactacgtactcattttggcaatatagatgcaactccagagattaaagaaatagcccatcgtttgacttacaaagctatccataagatgttggaattggaagttcccaacctgcatgagttagacctagatagggtgattgcttacggtcacacttggagtcccaccttggaacttgcgcctcgtctacccatgttccacggacacgccgttaatgtagatatggctttctcggcaacgatcgccgcccgtagaggatatattacaattgcagaacgcgatcgtattttaggattaatgagtcgcgttggtctatccctcgaccatcccatgttggatatagatattttgtggcgtggtactgaatctatcacattaactcgtgatggtttgttaagagctgctatgccaaaacccattggtgattgtgtcttcgtcaatgacctgacaagagaagaattagcagccgcattagctgaccacaaagaactttgtaccagttatccccgtggtggtgaaggtgtggatgtgtatcccgtttatcaaaaagaattaatcgggagtgttaaataatgacttttttgaattcaaaatgcaaaatactccacggatacactgcgcgagcgcggtagcatttctgttcgcggagcgtcccgtagggaaagagaaggctacgcaaataatcggacactaattgtctttaattttgaattttgaattttgaattttgaattggagcgaagcgacttgacaaatgtgattgtccaaccaacagctagacctgttacaccattgggaattttaaccaagcagttagaagccatagtccaagaggttaagcaacatccagatttacctggggaattgatagcaaacatccatcaggcttggcgtttagccgcaggtatagacccttatttggaagaatgcaccactccagaatctcctgaactcgctgcattggcaaaaaccacagccaccgaagcctggggagaacacttccacggaggtacaaccgtccgtcctctagaacaagagatgctttctggtcatatcgaaggacaaaccttaaagatgtttgttcacatgaccaaagctaaaaaagtcttagaaattgggatgtttaccggttattcggcgctggcgatggcggaagcattaccagaggatggactgcttgtggcttgtgaagttgacccttacgcggcggaaattggacagaaagcctttcaacaatctccccacggtggaaagattcgtgtggaattggatgcagccttagcaactcttgataagttagcagaagctggggagtcttttgacttggtatttatcgacgcagataaaaaagagtatgtagcctattttcacaagttgctaggtagcagtttgttagcaccagatggctttatttgtgtagataacaccttattacaaggggaagtttatctaccagcagaggaacgtagcgtcaatggtgaagcgatcgcgcaatttaatcatacagtagctatagacccccgtgtagaacaggttttgttgccgttgcgagatggtttaacaattatccgcagaatacaaccttaattgtccaatcgactatggcacaatcccttcccctttcttccgcacctgctacaccgtctcttccttcccagacgaaaatagccgcaattatccaaaatatctgcactttggctttgttattactagcattgcccattaatgccaccattgtttttatatccttgttagtcttccgaccgcaaaaggtcaaagcagcaaacccccaaaccattcttatcagtggcggtaagatgaccaaagctttacaactagcaaggtcattccacgcggctggacatagagttgtcttggtggaaacccataaatactggttgactggtcatcgtttttcccaagcagtggataagttttacacagtccccgcaccccaggacaatccccaagcttacattcaggctttggtagatatcgtcaaacaagaaaacatcgatgtttatattcccgtcaccagtccagtgggtagctactacgactcattagccaaaccagagttatcccattattgcgaagtgtttcactttgacgcagatattacccaaatgttggatgataaatttgcgttgacacaaaaagcgcgatcgcttggtttatcagtacccaaatcctttaaaattacctcaccagaacaagtcatcaacttcgatttttctggagagacacgtaaatacatcctcaaaagcattccctacgactcagtgcggcggttggacttaaccaaactcccctgtgctactccagaggaaacagcagcattcgtcagaagtttgccaattactcccgaaaaaccgtggattatgcaggaatttatccccggtaaggaattctgcacccatagcaccgttcggaatggggaactcagactgcattgctgttgcgaatcttcagccttccaagttaattatgagaatgtaaataacccgcaaattaccgaatgggtacagcattttgtcaaggaactgaaactgacaggacagatttcctttgactttatccaagccgaagacggaacagtttacgccatcgagtgtaacccccgcacacattcagcaattaccacattttacgaccacccccaggtagcagaagcgtacttgagtcaagcaccgacgactgaaaccatacaaccactaacgacaagcaagcctacctattggacttatcacgaagtttggcgtttaactggtatccgttctttcacccagttgcaaagatggctggggaatatttggcgcgggactgatgcgatttatcagccagatgaccccttaccgtttttgatggtacatcattggcaaattcccctactgttattgaataatttgcgtcgtcttaaaggttggacgcggatagatttcaatattgggaagttggtggaattggggggagattag

SEQ ID NO: 102 - DNA, 1233 bp, Anabaena variabilis ATCC29413
atgagtatcgtccaagcaaagtttgaagctaaggaaacatcttttcatgtagaaggttacgaaaagattgagtatgatttggtgtatgtagatggtatttttgaaatccagaattctgcactagcagatgtatatcaaggttttggacgatgcttggcgattgtagatgctaacgtcagtcggttgtatggtaatcaaattcaggcatatttccagtattatggtatagaactgaggctatttcctattaccattactgaaccagataagactattcaaactttcgagagagttatagatgtctttgcagatttcaaattagtccgcaaagaaccagtattagtcgtgggtggcggtttaattacagatgttgtcggctttgcttgttctacatatcgtcgcagcagcaattacatccgcattcctactacattgattggattaattgatgccagtgtagcaattaaggtagcagttaatcatcgcaaactgaaaaaccgtttgggtgcttatcatgcttctcgcaaagtatttttagatttctccttgttgcgtactctccctacagaccaagtacgtaacgggatggcggaattggtaaaaatcgctgtagtagcgcatcaagaagtttttgaattgttggagaagtacggcgaagaattactacgtactcattttggcaatatagatgcaactccagagattaaagaaatagcccatcgtttgacttacaaagctatccataagatgttggaattggaagttcccaacctgcatgagttagacctagatagggtgattgcttacggtcacacttggagtcccaccttggaacttgcgcctcgtctacccatgttccacggacacgccgttaatgtagatatggctttctcggcaacgatcgccgcccgtagaggatatattacaattgcagaacgcgatcgtattttaggattaatgagtcgcgttggtctatccctcgaccatcccatgttggatatagatattttgtggcgtggtactgaatctatcacattaactcgtgatggtttgttaagagctgctatgccaaaacccattggtgattgtgtcttcgtcaatgacctgacaagagaagaattagcagccgcattagctgaccacaaagaactttgtaccagttatccccgtggtggtgaaggtgtggatgtgtatcccgtttatcaaaaagaattaatcgggagtgttaaataa

SEQ ID NO: 103 - DNA, 840 bp, Anabaena variabilis ATCC29413
ttgacaaatgtgattgtccaaccaacagctagacctgttacaccattgggaattttaaccaagcagttagaagccatagtccaagaggttaagcaacatccagatttacctggggaattgatagcaaacatccatcaggcttggcgtttagccgcaggtatagacccttatttggaagaatgcaccactccagaatctcctgaactcgctgcattggcaaaaaccacagccaccgaagcctggggagaacacttccacggaggtacaaccgtccgtcctctagaacaagagatgctttctggtcatatcgaaggacaaaccttaaagatgtttgttcacatgaccaaagctaaaaaagtcttagaaattgggatgtttaccggttattcggcgctggcgatggcggaagcattaccagaggatggactgcttgtggcttgtgaagttgacccttacgcggcggaaattggacagaaagcctttcaacaatctccccacggtggaaagattcgtgtggaattggatgcagccttagcaactcttgataagttagcagaagctggggagtcttttgacttggtatttatcgacgcagataaaaaagagtatgtagcctattttcacaagttgctaggtagcagtttgttagcaccagatggctttatttgtgtagataacaccttattacaaggggaagtttatctaccagcagaggaacgtagcgtcaatggtgaagcgatcgcgcaatttaatcatacagtagctatagacccccgtgtagaacaggttttgttgccgttgcgagatggtttaacaattatccgcagaatacaaccttaa

SEQ ID NO: 104 - DNA, 1377 bp, Anabaena variabilis ATCC29413
atggcacaatcccttcccctttcttccgcacctgctacaccgtctcttccttcccagacgaaaatagccgcaattatccaaaatatctgcactttggctttgttattactagcattgcccattaatgccaccattgtttttatatccttgttagtcttccgaccgcaaaaggtcaaagcagcaaacccccaaaccattcttatcagtggcggtaagatgaccaaagctttacaactagcaaggtcattccacgcggctggacatagagttgtcttggtggaaacccataaatactggttgactggtcatcgtttttcccaagcagtggataagttttacacagtccccgcaccccaggacaatccccaagcttacattcaggctttggtagatatcgtcaaacaagaaaacatcgatgtttatattcccgtcaccagtccagtgggtagctactacgactcattagccaaaccagagttatcccattattgcgaagtgtttcactttgacgcagatattacccaaatgttggatgataaatttgcgttgacacaaaaagcgcgatcgcttggtttatcagtacccaaatcctttaaaattacctcaccagaacaagtcatcaacttcgatttttctggagagacacgtaaatacatcctcaaaagcattccctacgactcagtgcggcggttggacttaaccaaactcccctgtgctactccagaggaaacagcagcattcgtcagaagtttgccaattactcccgaaaaaccgtggattatgcaggaatttatccccggtaaggaattctgcacccatagcaccgttcggaatggggaactcagactgcattgctgttgcgaatcttcagccttccaagttaattatgagaatgtaaataacccgcaaattaccgaatgggtacagcattttgtcaaggaactgaaactgacaggacagatttcctttgactttatccaagccgaagacggaacagtttacgccatcgagtgtaacccccgcacacattcagcaattaccacattttacgaccacccccaggtagcagaagcgtacttgagtcaagcaccgacgactgaaaccatacaaccactaacgacaagcaagcctacctattggacttatcacgaagtttggcgtttaactggtatccgttctttcacccagttgcaaagatggctggggaatatttggcgcgggactgatgcgatttatcagccagatgaccccttaccgtttttgatggtacatcattggcaaattcccctactgttattgaataatttgcgtcgtcttaaaggttggacgcggatagatttcaatattgggaagttggtggaattggggggagattag

SEQ ID NO: 105 - DNA, 2358 bp, Anabaena variabilis ATCC29413
gttcctattgaccctgaatatcctcaagaacgcatagcttatatgctagaagattctcaggtgaaggtactactaactcaagaaaaattactcaatcaaattccccaccatcaagcacaaactatctgtgtagatagggaatgggagaaaatttccacacaagctaataccaatcccaaaagtaatataaaaacggataatcttgcttatgtaatttacacctctggttccactggtaaaccaaaaggtgcaatgaacacccacaaaggtatctgtaatcgcttattgtggatgcaggaagcttatcaaatcgattccacagatagcattttacaaaaaaccccctttagttttgatgtttccgtttgggagttcttttggactttattaactggcgcacgtttggtaatagccaaaccaggcggacataaagatagtgcttacctcatcgatttaattactcaagaacaaatcactacgttgcattttgtcccctcaatgctgcaagtgtttttacaaaatcgccatgtaagcaaatgcagctctctaaaaagagttatttgtagcggtgaagctttatctatagatttacaaaatagatttttccagcatttgcaatgtgaattacataacctctatggcccgacagaagcagcaattgatgtcacattttggcaatgtagaaaagatagtaatttaaagagtgtacctattggtcgtcccattgctaatactcaaatttatattcttgatgccgatttacaaccagtaaatattggtgtcactggtgaaatttatattggtggtgtaggggttgctcgtggttatttgaataaagaagaattgaccaaagaaaaatttattattaatccctttcccaattctgagtttaagcgactttataaaacaggtgatttagctcgttatttacccgatggaaatattgaatatcttggtagaacagattatcaagtaaaaattcggggttatagaattgaaattggcgagattgaaaatgttttatcttcacacccacaagtcagagaagctgtagtcatagcgcgggatgataacgctcaagaaaaacaaatcatcgcttatattacctataactccatcaaacctcagcttgataatctgcgtgatttcctaaaagcaaggctacctgattttatgattccagccgcttttgtgatgctggagcatcttcctttaactcccagtggtaaagtagaccgtaaggcattacctaagcctgatttatttaattatagtgaacataattcctatgtagcgcctcggaatgaagttgaagaaaaattagtacaaatctggtcgaatattctgcatttacctaaagtaggtgtgacagaaaactttttcgctattggtggtaattccctcaaagctctacatttaatttctcaaattgaagagttatttgctaaagagatatccttagcaacacttttaacaaatccagtaattgcagatttagccaaggttattcaagcaaacaaccaaatccataattcacccctagttccaattcaaccacaaggtaagcagcagcctttcttttgtatacatcctgctggtggtcatgttttatgctattttaaactcgcacaatatataggaactgaccaaccattttatggcttacaagctcaaggattttatggagatgaagcacccttgacgcgagttgaagatatggctagtctctacgtcaaaactattagagaatttcaaccccaagggccttatcgtgtcggggggtggtcatttggtggagtcgtagcttatgaagtagcacagcagttacatagacaaggacaagaagtatctttactagcaatattagattcttacgtaccgattctgctggataaacaaaaacccattgatgacgtttatttagttggtgttctctccagagtttttggcggtatgtttggtcaagataatctagtcacacctgaagaaatagaaaatttaactgtagaagaaaaaattaattacatcattgataaagcacggagcgctagaatattcccgcctggtgtagaacgtcaaaataatcgccgtattcttgatgttttggtgggaactttaaaagcaacttattcctatataagacaaccatatccaggaaaagtcactgtatttcgagccagggaaaaacatattatggctcctgacccgaccttagtttgggtagaattattttctgtaatggcggctcaagaaattaagattattgatgtccctggaaaccattattcgtttgttctagaaccccatgtacaggttttagcacagcgtttacaagattgtctggaaaataattcataa

SEQ ID NO: 106 - DNA, 1233 bp, Nostoc punctiforme ATCC29133
atgagtaatgttcaagcatcgtttgaagcaacggaagctgaattccgcgtggaaggttacgaaaaaattgagtttagtcttgtctatgtaaatggtgcatttgatatcagtaacagagaaattgcagacagctatgagaagtttggccgttgtctgactgtgattgatgctaatgtcaacagattatatggcaagcaaatcaagtcatattttagacactatggtattgatctgaccgtagttcccattgtgattactgagcctactaaaacccttgcaacctttgagaaaattgttgatgctttttctgactttggtttaatccgcaaggaaccagtattagtagtgggtggtggtttaaccactgatgtagctgggtttgcgtgtgctgcttaccgtcgtaagagtaactatattcgggttccgacaacgctgattggtctgattgatgcaggtgtagcgattaaggttgcagtcaaccatcgcaagttaaaaaatcgcttgggtgcatatcatgctcctttgaaagtcatcctcgatttctccttcttgcaaacattaccaacggctcaagttcgtaatgggatggcagagttggtcaaaattgctgttgtggcgaactctgaagtctttgaattgttgtatgaatatggcgaagagttgctttccactcactttggatatgtgaatggtacaaaggaactgaaagcgatcgcacataaactcaattacgaggcaataaaaactatgctggagttggaaactccaaacttgcatgagttagacctcgatcgcgtcattgcctacggtcacacttggagtccgaccttagaattagctccgatgataccgttgtttcacggtcatgccgtcaatatagacatggctttgtctgcaactattgcggcaagacgtggctacattacatcaggagaacgcgatcgcattttgagcttgatgagtcgtataggtttatcaatcgatcatcctctactagatggcgatttgctctggtatgctacccaatctattagcttgacgcgagacgggaaacaacgcgcagctatgcctaaacccattggtgagtgcttctttgtcaacgatttcacccgtgaagaactagatgcagctttagctgaacacaaacgtctgtgtgctacataccctcgtggtggagatggcattgacgcttacatagaaactcaagaagaatccaaactattgggagtgtga

SEQ ID NO: 107 - DNA, 834 bp, Nostoc punctiforme ATCC29133
atgaccagtattttaggacgagataccgcaagaccaataacgccacatagcattctggtagcacagctacaaaaaaccctcagaatggcagaggaaagtaatattccttcagagatactgacttctctgcgccaagggttgcaattagcagcaggtttagatccctatctggatgattgcactacccctgaatcgaccgcattgacagcactagcgcagaagacaagcattgaagactggagtaaacgcttcagtgatggtgaaacagtgcgtcaattagagcaagaaatgctctcaggacatcttgaaggacaaacactaaagatgtttgtgcatatcactaaggctaaaagcatcctagaagtgggaatgttcacaggatattcagctttggcaatggcagaggcgttaccagatgatgggcgactgattgcttgtgaagtagactcctatgtggccgagtttgctcaaacttgctttcaagagtctccccacggccgcaagattgttgtagaagtggcacctgcactagagacgctgcacaagctggtggctaaaaaagaatcctttgatctgatcttcattgatgcggataaaaaggagtatatagaatacttccaaattatcttggatagccatttactagctcccgacggattaatctgtgtagataatactttgttgcaaggacaagtttacctgccatcagaacagcgtacagccaatggtgaagcgatcgctcaattcaaccgcattgtcgccgcagatcctcgtgtagagcaagttctgttacccatacgagatggtataaccctgattagacgcttggtataa

SEQ ID NO: 108 - DNA, 1086 bp, Nostoc punctiforme ATCC29133
tactggttatcagggcatagattctcaaattctgtgagtcgtttttatacagttcctgcaccacaagacgacccagaaggctatacccaagcgctattggaaattgtcaaacgagagaagattgacgtttatgtacccgtatgcagccctgtagctagttattacgactctttggcaaagtctgcactatcagaatattgtgaggtttttcactttgatgctgatataaccaagatgctggatgataaatttgcctttaccgatcgggcgcgatcgcttggtttatcagccccgaaatcttttaaaattaccgatcctgaacaagttatcaacttcgattttagtaaagagacgcgcaaatatattcttaagagtatttcttacgactcagttcgccgcttaaatttaaccaaacttccttgtgataccccagaagagacagcagcgtttgtcaagagtttacccatcagcccagaaaaaccttggattatgcaagaatttattcctgggaaagaattatgcacccatagcacagtccgagacggcgaattaaggttgcattgctgttcaaattcttcagcgtttcagattaactatgaaaatgtcgaaaatccccaaattcaagaatgggtacaacatttcgtcaaaagtttacggctgactggacaaatatctcttgactttatccaagctgaagatggtacagcttatgccattgaatgtaatcctcgcacccattcggcgatcacaatgttctacaatcacccaggtgttgcagaagcctatcttggtaaaactcctctagctgcacctttggaacctcttgcagatagcaagcccacttactggatatatcacgaaatctggcgattgactgggattcgctctggacaacaattacaaacttggtttgggagattagtcagaggtacagatgccatttatcgcctggacgatccgataccatttttaactttgcaccattggcagattactttacttttgctacaaaatttgcaacgactcaaaggttgggtaaagatcgattttaatatcggtaaactcgtggaattagggggcgactaa

SEQ ID NO: 109 - DNA, 1047 bp, Nostoc punctiforme ATCC29133
atgccagtacttaatatccttcatttagttgggtctgcacacgataagttttactgtgatttatcacgtctttatgcccaagactgtttagctgcaacagcagatccatcgctttataactttcaaattgcatatatcacacccgatcggcagtggcgatttcctgactctctcagtcgagaagatattgctcttaccaaaccgattcctgtgtttgatgccatacaatttctaacaggccaaaacattgacatgatgttaccacaaatgttttgtattcctggaatgactcagtaccgtgccctattcgatctgctcaagatcccttatataggaaataccccagatattatggcgatcgcggcccacaaagccagagccaaagcaattgtcgaagcagcaggggtaaaagtgcctcgtggagaattgcttcgccaaggagatattccaacaattacacctccagcagtcgtcaaacctgtaagttctgacaactctttaggagtagtcttagttaaagatgtgactgaatatgatgctgccttaaagaaagcatttgaatatgcttcggaggtcatcgtagaagcattcatcgaacttggtcgagaagtcagatgcggcatcattgtaaaagacggtgagctaataggtttaccccttgaagagtatctggtagacccacacgataaacctatccgtaactatgctgataaactccaacaaactgacgatggcgacttgcatttgactgctaaagataatatcaaggcttggattttagaccctaacgacccaatcacccaaaaggttcagcaagtggctaaaaggtgtcatcaggctttgggttgtcgccactacagtttatttgacttccgaatcgatccaaagggacaaccttggttcttagaagctggattatattgttcttttgcccccaaaagtgtgatttcttctatggcgaaagcagccggaatccctctaaatgatttattaataaccgctattaatgaaacattgggtagtaataaaaaggtgttacaaaattga

SEQ ID NO: 110 - DNA, 1626 bp, Saccharomyces cerevisiae CEN.PK-1D
aacaagccgggctttaccttgtctgacaactacacctatgttttcttgggtgacggttgtttgcaagaaggtatttcttcagaagcttcctccttggctggtcatttgaaattgggtaacttgattgccatctacgatgacaacaagatcactatcgatggtgctaccagtatctcattcgatgaagatgttgctaagagatacgaagcctacggttgggaagttttgtacgtagaaaatggtaacgaagatctagccggtattgccaaggctattgctcaagctaagttatccaaggacaaaccaactttgatcaaaatgaccacaaccattggttacggttccttgcatgccggctctcactctgtgcacggtgccccattgaaagcagatgatgttaaacaactaaagagcaaattcggtttcaacccagacaagtcctttgttgttccacaagaagtttacgaccactaccaaaagacaattttaaagccaggtgtcgaagccaacaacaagtggaacaagttgttcagcgaataccaaaagaaattcccagaattaggtgctgaattggctagaagattgagcggccaactacccgcaaattgggaatctaagttgccaacttacaccgccaaggactctgccgtggccactagaaaattatcagaaactgttcttgaggatgtttacaatcaattgccagagttgattggtggttctgccgatttaacaccttctaacttgaccagatggaaggaagcccttgacttccaacctccttcttccggttcaggtaactactctggtagatacattaggtacggtattagagaacacgctatgggtgccataatgaacggtatttcagctttcggtgccaactacaaaccatacggtggtactttcttgaacttcgtttcttatgctgctggtgccgttagattgtccgctttgtctggccacccagttatttgggttgctacacatgactctatcggtgtcggtgaagatggtccaacacatcaacctattgaaactttagcacacttcagatccctaccaaacattcaagtttggagaccagctgatggtaacgaagtttctgccgcctacaagaactctttagaatccaagcatactccaagtatcattgctttgtccagacaaaacttgccacaattggaaggtagctctattgaaagcgcttctaagggtggttacgtactacaagatgttgctaacccagatattattttagtggctactggttccgaagtgtctttgagtgttgaagctgctaagactttggccgcaaagaacatcaaggctcgtgttgtttctctaccagatttcttcacttttgacaaacaacccctagaatacagactatcagtcttaccagacaacgttccaatcatgtctgttgaagttttggctaccacatgttggggcaaatacgctcatcaatccttcggtattgacagatttggtgcctccggtaaggcaccagaagtcttcaagttcttcggtttcaccccagaaggtgttgctgaaagagctcaaaagaccattgcattctataagggtgacaagctaatttctcctttgaaaaaagctttctaa

SEQ ID NO: 111 - DNA, 1113 bp, Saccharomyces cerevisiae CEN.PK-1D
atgagtgaatctccaatgttcgctgccaacggcatgccaaaggtaaatcaaggtgctgaagaagatgtcagaattttaggttacgacccattagcttctccagctctccttcaagtgcaaatcccagccacaccaacttctttggaaactgccaagagaggtagaagagaagctatagatattattaccggtaaagacgacagagttcttgtcattgtcggtccttgttccatccatgatctagaagccgctcaagaatacgctttgagattaaagaaattgtcagatgaattaaaaggtgatttatccatcattatgagagcatacttggagaagccaagaacaaccgtcggctggaaaggtctaattaatgaccctgatgttaacaacactttcaacatcaacaagggtttgcaatccgctagacaattgtttgtcaacttgacaaatatcggtttgccaattggttctgaaatgcttgataccatttctcctcaatacttggctgatttggtctccttcggtgccattggtgccagaaccaccgaatctcaactgcacagagaattggcctccggtttgtctttcccagttggtttcaagaacggtaccgatggtaccttaaatgttgctgtggatgcttgtcaagccgctgctcattctcaccatttcatgggtgttactaagcatggtgttgctgctatcaccactactaagggtaacgaacactgcttcgttattctaagaggtggtaaaaagggtaccaactacgacgctaagtccgttgcagaagctaaggctcaattgcctgccggttccaacggtctaatgattgactactctcacggtaactccaataaggatttcagaaaccaaccaaaggtcaatgacgttgtttgtgagcaaatcgctaacggtgaaaacgccattaccggtgtcatgattgaatcaaacatcaacgaaggtaaccaaggcatcccagccgaaggtaaagccggcttgaaatatggtgtttccatcactgatgcttgtataggttgggaaactactgaagacgtcttgaggaaattggctgctgctgtcagacaaagaagagaagttaacaagaaatag

SEQ ID NO: 112 - protein, 1588 aa, Saccharomyces cerevisiae
MVQLAKVPILGNDIIHVGYNIHDHLVETIIKHCPSSTYVICNDTNLSKVPYYQQLVLEFKASLPEGSRLLTYVVKPGETSKSRETKAQLEDYLLVEGCTRDTVMVAIGGGVIGDMIGFVASTFMRGVRVVQVPTSLLAMVDSSIGGKTAIDTPLGKNFIGAFWQPKFVLVDIKWLETLAKREFINGMAEVIKTACIWNADEFTRLESNASLFLNVVNGAKNVKVTNQLTNEIDEISNTDIEAMLDHTYKLVLESIKVKAEVVSSDERESSLRNLLNFGHSIGHAYEAILTPQALHGECVSIGMVKEAELSRYFGILSPTQVARLSKILVAYGLPVSPDEKWFKELTLHKKTPLDILLKKMSIDKKNEGSKKKVVILESIGKCYGDSAQFVSDEDLRFILTDETLVYPFKDIPADQQKVVIPPGSKSISNRALILAALGEGQCKIKNLLHSDDTKHMLTAVHELKGATISWEDNGETVVVEGHGGSTLSACADPLYLGNAGTASRFLTSLAALVNSTSSQKYIVLTGNARMQQRPIAPLVDSLRANGTKIEYLNNEGSLPIKVYTDSVFKGGRIELAATVSSQYVSSILMCAPYAEEPVTLALVGGKPISKLYVDMTIKMMEKFGINVETSTTEPYTYYIPKGHYINPSEYVIESDASSATYPLAFAAMTGTTVTVPNIGFESLQGDARFARDVLKPMGCKITQTATSTTVSGPPVGTLKPLKHVDMEPMTDAFLTACVVAAISHDSDPNSANTTTIEGIANQRVKECNRILAMATELAKFGVKTTELPDGIQVHGLNSIKDLKVPSDSSGPVGVCTYDDHRVAMSFSLLAGMVNSQNERDEVANPVRILERHCTGKTWPGWWDVLHSELGAKLDGAEPLECTSKKNSKKSVVIIGMRAAGKTTISKWCASALGYKLVDLDELFEQQHNNQSVKQFVVENGWEKFREEETRIFKEVIQNYGDDGYVFSTGGGIVESAESRKALKDFASSGGYVLHLHRDIEETIVFLQSDPSRPAYVEEIREVWNRREGWYKECSNFSFFAPHCSAEAEFQALRRSFSKYIATITGVREIEIPSGRSAFVCLTFDDLTEQTENLTPICYGCEAVEVRVDHLANYSADFVSKQLSILRKATDSIPIIFTVRTMKQGGNFPDEEFKTLRELYDIALKNGVEFLDLELTLPTDIQYEVINKRGNTKIIGSHHDFQGLYSWDDAEWENRFNQALTLDVDVVKFVGTAVNFEDNLRLEHFRDTHKNKPLIAVNMTSKGSISRVLNNVLTPVTSDLLPNSAAPGQLTVAQINKMYTSMGGIEPKELFVVGKPIGHSRSPILHNTGYEILGLPHKFDKFETESAQLVKEKLLDGNKNFGGAAVTIPLKLDIMQYMDELTDAAKVIGAVNTVIPLGNKKFKGDNTDWLGIRNALINNGVPEYVGHTAGLVIGAGGTSRAALYALHSLGCKKIFIINRTTSKLKPLIESLPSEFNIIGIESTKSIEEIKEHVGVAVSCVPADKPLDDELLSKLERFLVKGAHAAFVPTLLEAAYKPSVTPVMTISQDKYQWHVVPGSQMLVHQGVAQFEKWTGFKGPFKAIFDAVTKE

SEQ ID NO: 113 - protein, 820 aa, Anabaena variabilis ATCC29413
MSIVQAKFEAKETSFHVEGYEKIEYDLVYVDGIFEIQNSALADVYQGFGRCLAIVDANVSRLYGNQIQAYFQYYGIELRLFPITITEPDKTIQTFERVIDVFADFKLVRKEPVLVVGGGLITDVVGFACSTYRRSSNYIRIPTTLIGLIDASVAIKVAVNHRKLKNRLGAYHASRKVFLDFSLLRTLPTDQVRNGMAELVKIAVVAHQEVFELLEKYGEELLRTHFGNIDATPEIKEIAHRLTYKAIHKMLELEVPNLHELDLDRVIAYGHTWSPTLELAPRLPMFHGHAVNVDMAFSATIAARRGYITIAERDRILGLMSRVGLSLDHPMLDIDILWRGTESITLTRDGLLRAAMPKPIGDCVFVNDLTREELAAALADHKELCTSYPRGGEGVDVYPVYQKELIGSVKMSIVQAKFEAKETSFHVEGYEKIEYDLVYVDGIFEIQNSALADVYQGFGRCLAIVDANVSRLYGNQIQAYFQYYGIELRLFPITITEPDKTIQTFERVIDVFADFKLVRKEPVLVVGGGLITDVVGFACSTYRRSSNYIRIPTTLIGLIDASVAIKVAVNHRKLKNRLGAYHASRKVFLDFSLLRTLPTDQVRNGMAELVKIAVVAHQEVFELLEKYGEELLRTHFGNIDATPEIKEIAHRLTYKAIHKMLELEVPNLHELDLDRVIAYGHTWSPTLELAPRLPMFHGHAVNVDMAFSATIAARRGYITIAERDRILGLMSRVGLSLDHPMLDIDILWRGTESITLTRDGLLRAAMPKPIGDCVFVNDLTREELAAALADHKELCTSYPRGGEGVDVYPVYQKELIGSVK

SEQ ID NO: 114 - protein, 1099 aa, Anabaena variabilis ATCC29413
MSIVQAKFEAKETSFHVEGYEKIEYDLVYVDGIFEIQNSALADVYQGFGRCLAIVDANVSRLYGNQIQAYFQYYGIELRLFPITITEPDKTIQTFERVIDVFADFKLVRKEPVLVVGGGLITDVVGFACSTYRRSSNYIRIPTTLIGLIDASVAIKVAVNHRKLKNRLGAYHASRKVFLDFSLLRTLPTDQVRNGMAELVKIAVVAHQEVFELLEKYGEELLRTHFGNIDATPEIKEIAHRLTYKAIHKMLELEVPNLHELDLDRVIAYGHTWSPTLELAPRLPMFHGHAVNVDMAFSATIAARRGYITIAERDRILGLMSRVGLSLDHPMLDIDILWRGTESITLTRDGLLRAAMPKPIGDCVFVNDLTREELAAALADHKELCTSYPRGGEGVDVYPVYQKELIGSVKMSIVQAKFEAKETSFHVEGYEKIEYDLVYVDGIFEIQNSALADVYQGFGRCLAIVDANVSRLYGNQIQAYFQYYGIELRLFPITITEPDKTIQTFERVIDVFADFKLVRKEPVLVVGGGLITDVVGFACSTYRRSSNYIRIPTTLIGLIDASVAIKVAVNHRKLKNRLGAYHASRKVFLDFSLLRTLPTDQVRNGMAELVKIAVVAHQEVFELLEKYGEELLRTHFGNIDATPEIKEIAHRLTYKAIHKMLELEVPNLHELDLDRVIAYGHTWSPTLELAPRLPMFHGHAVNVDMAFSATIAARRGYITIAERDRILGLMSRVGLSLDHPMLDIDILWRGTESITLTRDGLLRAAMPKPIGDCVFVNDLTREELAAALADHKELCTSYPRGGEGVDVYPVYQKELIGSVKMTNVIVQPTARPVTPLGILTKQLEAIVQEVKQHPDLPGELIANIHQAWRLAAGIDPYLEECTTPESPELAALAKTTATEAWGEHFHGGTTVRPLEQEMLSGHIEGQTLKMFVHMTKAKKVLEIGMFTGYSALAMAEALPEDGLLVACEVDPYAAEIGQKAFQQSPHGGKIRVELDAALATLDKLAEAGESFDLVFIDADKKEYVAYFHKLLGSSLLAPDGFICVDNTLLQGEVYLPAEERSVNGEAIAQFNHTVAIDPRVEQVLLPLRDGLTIIRRIQP

SEQ ID NO: 115 - protein, 410 aa, Anabaena variabilis ATCC29413
MSIVQAKFEAKETSFHVEGYEKIEYDLVYVDGIFEIQNSALADVYQGFGRCLAIVDANVSRLYGNQIQAYFQYYGIELRLFPITITEPDKTIQTFERVIDVFADFKLVRKEPVLVVGGGLITDVVGFACSTYRRSSNYIRIPTTLIGLIDASVAIKVAVNHRKLKNRLGAYHASRKVFLDFSLLRTLPTDQVRNGMAELVKIAVVAHQEVFELLEKYGEELLRTHFGNIDATPEIKEIAHRLTYKAIHKMLELEVPNLHELDLDRVIAYGHTWSPTLELAPRLPMFHGHAVNVDMAFSATIAARRGYITIAERDRILGLMSRVGLSLDHPMLDIDILWRGTESITLTRDGLLRAAMPKPIGDCVFVNDLTREELAAALADHKELCTSYPRGGEGVDVYPVYQKELIGSVK

SEQ ID NO: 116 - protein, 410 aa, Anabaena variabilis ATCC29413; identical to SEQ ID NO: 115

SEQ ID NO: 117 - protein, 279 aa, Anabaena variabilis ATCC29413
MTNVIVQPTARPVTPLGILTKQLEAIVQEVKQHPDLPGELIANIHQAWRLAAGIDPYLEECTTPESPELAALAKTTATEAWGEHFHGGTTVRPLEQEMLSGHIEGQTLKMFVHMTKAKKVLEIGMFTGYSALAMAEALPEDGLLVACEVDPYAAEIGQKAFQQSPHGGKIRVELDAALATLDKLAEAGESFDLVFIDADKKEYVAYFHKLLGSSLLAPDGFICVDNTLLQGEVYLPAEERSVNGEAIAQFNHTVAIDPRVEQVLLPLRDGLTIIRRIQP

SEQ ID NO: 118 - protein, 888 aa, Anabaena variabilis ATCC29413
MQTIDFNIRKLLVEWNATHRDYDLSQSLHELIVAQVERTPEAIAVTFDKQQLTYQELNHKANQLGHYLQTLGVQPETLVGVCLERSLEMVICLLGILKAGGAYVPIDPEYPQERIAYMLEDSQVKVLLTQEKLLNQIPHHQAQTICVDREWEKISTQANTNPKSNIKTDNLAYVIYTSGSTGKPKGAMNTHKGICNRLLWMQEAYQIDSTDSILQKTPFSFDVSVWEFFWTLLTGARLVIAKPGGHKDSAYLIDLITQEQITTLHFVPSMLQVFLQNRHVSKCSSLKRVICSGEALSIDLQNRFFQHLQCELHNLYGPTEAAIDVTFWQCRKDSNLKSVPIGRPIANTQIYILDADLQPVNIGVTGEIYIGGVGVARGYLNKEELTKEKFIINPFPNSEFKRLYKTGDLARYLPDGNIEYLGRTDYQVKIRGYRIEIGEIENVLSSHPQVREAVVIARDDNAQEKQIIAYITYNSIKPQLDNLRDFLKARLPDFMIPAAFVMLEHLPLTPSGKVDRKALPKPDLFNYSEHNSYVAPRNEVEEKLVQIWSNILHLPKVGVTENFFAIGGNSLKALHLISQIEELFAKEISLATLLTNPVIADLAKVIQANNQIHNSPLVPIQPQGKQQPFFCIHPAGGHVLCYFKLAQYIGTDQPFYGLQAQGFYGDEAPLTRVEDMASLYVKTIREFQPQGPYRVGGWSFGGVVAYEVAQQLHRQGQEVSLLAILDSYVPILLDKQKPIDDVYLVGVLSRVFGGMFGQDNLVTPEEIENLTVEEKINYIIDKARSARIFPPGVERQNNRRILDVLVGTLKATYSYIRQPYPGKVTVFRAREKHIMAPDPTLVWVELFSVMAAQEIKIIDVPGNHYSFVLEPHVQVLAQRLQDCLENNS

SEQ ID NO: 119 - protein, 410 aa, Nostoc punctiforme ATCC 29133
MSNVQASFEATEAEFRVEGYEKIEFSLVYVNGAFDISNREIADSYEKFGRCLTVIDANVNRLYGKQIKSYFRHYGIDLTVVPIVITEPTKTLATFEKIVDAFSDFGLIRKEPVLVVGGGLTTDVAGFACAAYRRKSNYIRVPTTLIGLIDAGVAIKVAVNHRKLKNRLGAYHAPLKVILDFSFLQTLPTAQVRNGMAELVKIAVVANSEVFELLYEYGEELLSTHFGYVNGTKELKAIAHKLNYEAIKTMLELETPNLHELDLDRVIAYGHTWSPTLELAPMIPLFHGHAVNIDMALSATIAARRGYITSGERDRILSLMSRIGLSIDHPLLDGDLLWYATQSISLTRDGKQRAAMPKPIGECFFVNDFTREELDAALAEHKRLCATYPRGGDGIDAYIETQEESKLLGV

SEQ ID NO: 120 - protein, 277 aa, Nostoc punctiforme ATCC 29133
MTSILGRDTARPITPHSILVAQLQKTLRMAEESNIPSEILTSLRQGLQLAAGLDPYLDDCTTPESTALTALAQKTSIEDWSKRFSDGETVRQLEQEMLSGHLEGQTLKMFVHITKAKSILEVGMFTGYSALAMAEALPDDGRLIACEVDSYVAEFAQTCFQESPHGRKIVVEVAPALETLHKLVAKKESFDLIFIDADKKEYIEYFQIILDSHLLAPDGLICVDNTLLQGQVYLPSEQRTANGEAIAQFNRIVAADPRVEQVLLPIRDGITLIRRLV

SEQ ID NO: 121 - protein, 461 aa, Nostoc punctiforme ATCC 29133
MAQSISLSLPQSTTPSKGVRLKIAALLKTIGTLILLLIALPLNALIVLISLMCRPFTKKPAVATHPQNILVSGGKMTKALQLARSFHAAGHRVILIEGHKYWLSGHRFSNSVSRFYTVPAPQDDPEGYTQALLEIVKREKIDVYVPVCSPVASYYDSLAKSALSEYCEVFHFDADITKMLDDKFAFTDRARSLGLSAPKSFKITDPEQVINFDFSKETRKYILKSISYDSVRRLNLTKLPCDTPEETAAFVKSLPISPEKPWIMQEFIPGKELCTHSTVRDGELRLHCCSNSSAFQINYENVENPQIQEWVQHFVKSLRLTGQISLDFIQAEDGTAYAIECNPRTHSAITMFYNHPGVAEAYLGKTPLAAPLEPLADSKPTYWIYHEIWRLTGIRSGQQLQTWFGRLVRGTDAIYRLDDPIPFLTLHHWQITLLLLQNLQRLKGWVKIDFNIGKLVELGGD

SEQ ID NO: 122 - protein, 348 aa, Nostoc punctiforme ATCC 29133
MPVLNILHLVGSAHDKFYCDLSRLYAQDCLAATADPSLYNFQIAYITPDRQWRFPDSLSREDIALTKPIPVFDAIQFLTGQNIDMMLPQMFCIPGMTQYRALFDLLKIPYIGNTPDIMAIAAHKARAKAIVEAAGVKVPRGELLRQGDIPTITPPAVVKPVSSDNSLGVVLVKDVTEYDAALKKAFEYASEVIVEAFIELGREVRCGIIVKDGELIGLPLEEYLVDPHDKPIRNYADKLQQTDDGDLHLTAKDNIKAWILDPNDPITQKVQQVAKRCHQALGCRHYSLFDFRIDPKGQPWFLEAGLYCSFAPKSVISSMAKAAGIPLNDLLITAINETLGSNKKVLQN

SEQ ID NO: 123 - protein, 680 aa, Saccharomyces cerevisiae CEN.PK-1D
MTQFTDIDKLAVSTIRILAVDTVSKANSGHPGAPLGMAPAAHVLWSQMRMNPTNPDWINRDRFVLSNGHAVALLYSMLHLTGYDLSIEDLKQFRQLGSRTPGHPEFELPGVEVTTGPLGQGISNAVGMAMAQANLAATYNKPGFTLSDNYTYVFLGDGCLQEGISSEASSLAGHLKLGNLIAIYDDNKITIDGATSISFDEDVAKRYEAYGWEVLYVENGNEDLAGIAKAIAQAKLSKDKPTLIKMTTTIGYGSLHAGSHSVHGAPLKADDVKQLKSKFGFNPDKSFVVPQEVYDHYQKTILKPGVEANNKWNKLFSEYQKKFPELGAELARRLSGQLPANWESKLPTYTAKDSAVATRKLSETVLEDVYNQLPELIGGSADLTPSNLTRWKEALDFQPPSSGSGNYSGRYIRYGIREHAMGAIMNGISAFGANYKPYGGTFLNFVSYAAGAVRLSALSGHPVIWVATHDSIGVGEDGPTHQPIETLAHFRSLPNIQVWRPADGNEVSAAYKNSLESKHTPSIIALSRQNLPQLEGSSIESASKGGYVLQDVANPDIILVATGSEVSLSVEAAKTLAAKNIKARVVSLPDFFTFDKQPLEYRLSVLPDNVPIMSVEVLATTCWGKYAHQSFGIDRFGASGKAPEVFKFFGFTPEGVAERAQKTIAFYKGDKLISPLKKAF

SEQ ID NO: 124 - protein, 370 aa, Saccharomyces cerevisiae CEN.PK-1D
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

(19) United States
(12) Patent Application Publication
(10) Pub. No.: US 2021/0230610 A1
(43) Pub. Date: Jul. 29, 2021

(54) A MICROORGANISM PRODUCING A MYCOSPORINE-LIKE AMINO ACID AND A METHOD FOR PRODUCING A MYCOSPORINE-LIKE AMINO ACID USING THE SAME

(71) Applicant: CJ CHEILJEDANG CORPORATION, Seoul (KR)

(72) Inventors: Sol Kim, Seoul (KR); Jong-cheol Seok, Seoul (KR); Kyusung Lee, Seoul (KR); Jae Woo Jang, Seoul (KR)

(73) Assignee: CJ CHEILJEDANG CORPORATION, Seoul (KR)

(21) Appl. No.: 16/971,209

(22) PCT Filed: Feb. 22, 2019
(86) PCT No.: PCT/KR2019/002242

§ 371 Date: Jan. 6, 2021

(30) Foreign Application Priority Data
Feb. 23, 2018 (KR) 10-2018-0022185

(51) Int. Cl. C12N 15/81; C12N 15/70; C12N 15/77; C12N 9/10; C12N 9/12

(52) U.S. Cl. CPC C12N 15/81; C12N 15/70; C12N 15/77; C12N 9/1022; C12Y 207/09002; C12N 9/1294; C12Y 202/01001; C12Y 205/01054; C12N 9/1085

(57) ABSTRACT

The present disclosure relates to a microorganism producing a mycosporine-like amino acid and a method for producing a mycosporine-like amino acid using the microorganism.

Since the microorganism of the present disclosure has an increased mycosporine-like amino acid-producing ability, it can be effectively used in producing mycosporine-like amino acids.

### TECHNICAL FIELD

The present disclosure relates to a microorganism producing a mycosporine-like amino acid, and a method for producing a mycosporine-like amino acid using the microorganism.

### BACKGROUND ART

The ultraviolet (UV) rays emitted by the sun consists of UVA (with a wavelength of about 320 nm to about 400 nm), UVB (with a wavelength of about 290 nm to about 320 nm), and UVC (with a wavelength of about 100 nm to about 280 nm). It is known that UVA rays penetrate into the dermal layer, mainly induce pigmentation and aging of the skin, and are involved in the occurrence of photosensitive skin disease, whereas UVB rays are high-energy rays that penetrate the epidermis and the basal layer of the dermis and are involved in sunburn, pigmentation, and occurrence of skin cancer.

To prevent these side effects of UV rays, attempts have been made to block UV rays. The types of sunscreen agents include chemical sunscreen agents and physical sunscreen agents. Chemical sunscreen agents prevent the penetration of UV rays mainly by absorption of UV rays, whereas physical sunscreen agents prevent the penetration of UV rays through reflection and scattering.

Components that are known to be contained in the chemical sunscreen agents may include those which mainly absorb UVB rays (e.g., PABA, PABA esters (amyl dimethyl PABA, octyl dimethyl PABA), cinnamates (cinoxate), salicylates (homomenthyl salicylate), camphor, etc.); and those which mainly absorb UVA rays (e.g., benzophenones (oxybenzone, dioxybenzone, and suliso benzene), dibenzoyl methane, anthranilate, etc.). Although these chemical sunscreen agents can provide effects of absorbing and blocking UV rays, it is known that some of these chemical sunscreen agents can irritate the skin or eyes, and in particular, PABA, PABA esters, benzophenones, and cinnamates, etc. can cause contact dermatitis. Additionally, some others of these chemical sunscreen agents have been reported to cause a photosensitive reaction in the skin, etc. Accordingly, in some countries, the use or the amount of use of these chemical sunscreen agents is being limited.

Components that are known to be contained in the physical sunscreen agents may include titanium dioxide, talc (magnesium silicate), magnesium oxide, zinc oxide, kaolin, etc. The physical sunscreen agents have advantages in that they do not cause side effects, such as contact dermatitis and that they are not easily removed by water. However, they have disadvantages in that it is difficult for them to maintain an effective content while realizing desired formulations and that a white cast, etc. occur when they are applied to the skin.

Mycosporine-like amino acids (MAAs) are materials present in natural organisms and they are known to absorb UVA and UVB effectively. More than 35 species of MAAs are known to be present in nature (Mar. Biol., 1991, 108: 157-166; Planta Med., 2015, 81: 813-820). Recently, MAAs, to which various kinds of sugars are attached, have been reported to exist in microalgae and they have an excellent antioxidant function (Journal of Photochemistry and Photobiology, 2015, 142: 154-168). Additionally, MAAs are known to provide not only an ability blocking UV rays, but also resistance to oxidation, osmosis, heat stress, etc. (Comp. Biochem. Physiol. C. Toxicol. Pharmacol., 2007, 146: 60-78; J. Photochem. Photobiol. B., 2007, 89: 29-35).

However, the amount of MAAs produced within microalgae is very low to be at the level of several micrograms, and the conditions for separation, extraction, and purification of MAAs after culturing microalgae are complicated. Therefore, it is difficult to mass-produce the MAAs material.

### PRIOR ART DOCUMENTS

### Non-Patent Documents

(Non-patent Document 1) Comp. Biochem. Physiol. B 1995, 112: 105-114.
(Non-patent Document 2) FEMS Microbiol Lett. 2007, 269: 1-10.
(Non-patent Document 3) Ann. Rev. Physiol. 2002, 64: 223-262.
(Non-patent Document 4)Mar. Biol. 1991, 108: 157-166.
(Non-patent Document 5) Journal of Photochemistry and Photobiology B: Biology. 2015, 142: 154-168
(Non-patent Document 6) Biol. Rev. 1999, 74: 311-345.
(Non-patent Document 7) Mol. Biol. Evol. 2006, 23: 1437-1443.
(Non-patent Document 8) Science, 2010, 329: 1653-1656.
(Non-patent Document 9) Genomics 2010, 95: 120-128.
(Non-patent Document 10) Geomicrobiol. J. 1997. 14: 231-241.
(Non-patent Document 11) Comp. Biochem. Physiol. C Toxicol. Pharmacol. 2007. 146: 60-78.
(Non-patent Document 12) Can. J. Bot. 2003. 81: 131-138.
(Non-patent Document 13) J. Photochem. Photobiol. B. 2007, 89: 29-35.
(Non-patent Document 14)J. Bacteriol. 2011. 193(21): 5923-5928.
(Non-patent Document 15) Planta Med. 2015. 81: 813-820
(Non-patent Document 16) ACS Appl. Mater. Interfaces. 2015. 7: 16558-16564
(Non-patent Document 17) Appl Environ Microbiol. 2016, 82(20): 6167-6173
(Non-patent Document 18) ChemBioChem. 2015, 16: 320-327
(Non-patent Document 19) Methods Mol Biol. 2013, 1073: 43-7
(Non-patent Document 20) Nature Review, 2011, 9: 791-802

### DISCLOSURE

### Technical Problem

The present inventors have made many efforts to increase the production of MAAs in microorganisms. As a result, they have confirmed that the production of MAAs can be increased in microorganisms producing MAAs through various studies associated with the enhancement of the activity of 2-dehydro-3-deoxyphosphoheptonate aldolase, phosphoenolpyruvate synthetase, and transketolase proteins in microorganisms, thereby completing the present disclosure.

### Technical Solution

An object of the present disclosure is to provide a microorganism producing a mycosporine-like amino acid (MMA), in which an activity of at least one protein selected from the group consisting of 2-dehydro-3-deoxyphosphoheptonate aldolase, phosphoenolpyruvate synthetase, and transketolase is enhanced.

Another object of the present disclosure is to provide a method for producing a mycosporine-like amino acid, which includes culturing the microorganism in a medium; and recovering the mycosporine-like amino acid from the cultured microorganism or medium.

Still another object of the present disclosure is to provide a use of the microorganism for producing a mycosporine-like amino acid.

### Advantageous Effects

Since the microorganism of the present disclosure has an increased mycosporine-like amino acid (MMA)-producing ability, it can be effectively used in producing mycosporine-like amino acids.

### BEST MODE FOR CARRYING OUT THE INVENTION

The present disclosure is described in detail hereinbelow. Meanwhile, respective descriptions and embodiments disclosed in the present disclosure may also be applied to other descriptions and embodiments. That is, all combinations of various elements disclosed in the present disclosure fall within the scope of the present disclosure. In addition, the scope of the present disclosure is not limited by the specific description below. In addition, one of ordinary skill in the art can recognize or identify a number of equivalents with regard to certain aspects of the present disclosure only by routine experimentation. Further, such equivalents are intended to be included in the present disclosure.

To achieve the above objects, an aspect of the present disclosure provides a microorganism producing a mycosporine-like amino acid (MMA), in which an activity of at least one protein selected from the group consisting of 2-dehydro-3-deoxyphosphoheptonate aldolase, phosphoenolpyruvate synthetase, and transketolase is enhanced.

As used herein, the term “2-dehydro-3-deoxyphosphoheptonate aldolase” refers to an enzyme that catalyzes the reversible reaction of the following reaction scheme, and specifically, may refer to an enzyme that synthesizes 3-deoxy-D-arabino-heptulosonate-7-phosphate (DAHP), but is not limited thereto.

phosphoenolpyruvate+D-erythrose-4-phosphate+H₂O↔3-deoxy-D-arabinoheptulosonate-7-phosphate+phosphate [Reaction Scheme]

In the present disclosure, 2-dehydro-3-deoxyphosphoheptonate aldolase can be used interchangeably with 3-deoxy-D-arabino-heptulosonate-7-phosphate (DAHP) synthase.

As used herein, the term “phosphoenolpyruvate synthetase” refers to an enzyme that catalyzes the reversible reaction of the following reaction scheme, and specifically, may refer to an enzyme that synthesizes phosphoenolpyruvate, but is not limited thereto.

ATP+pyruvate+H₂O↔AMP+phosphoenolpyruvate+phosphate [Reaction Scheme]

As used herein, the term “transketolase” refers to an enzyme that catalyzes the reversible reaction of the following reaction scheme.

sedoheptulose 7-phosphate+D-glyceraldehyde 3-phosphate↔D-ribose 5-phosphate+D-xylulose 5-phosphate

or

fructose 6-phosphate+D-glyceraldehyde 3-phosphate↔erythrose 4-phosphate+D-xylulose 5-phosphate [Reaction Scheme]

The genetic information of the 2-dehydro-3-deoxyphosphoheptonate aldolase, phosphoenolpyruvate synthetase, and transketolase can be obtained from known database (e.g., GenBank database of the National Center for Biotechnology Information (NCBI), etc.), but is not limited thereto.

The 2-dehydro-3-deoxyphosphoheptonate aldolase, phosphoenolpyruvate synthetase, and transketolase are not limited to their origins or sequences because there are cases where the proteins showing the activities differ in their amino acid sequences depending on the species of a microorganism or the microorganism itself.

Specifically, the 2-dehydro-3-deoxyphosphoheptonate aldolase may be a protein including the amino acid sequence of SEQ ID NO: 2, 37, or 124; the phosphoenolpyruvate synthetase may be a protein including the amino acid sequence of SEQ ID NO: 19 or 98; and the transketolase may be a protein including the amino acid sequence of SEQ ID NO: 24, 96, or 123, but the amino acid sequences of these proteins are not limited thereto. As used herein, the term “a protein including an amino acid sequence” can be used interchangeably with the expression of “a protein having an amino acid sequence” or “a protein consisting of an amino acid sequence”.

Additionally, in the present disclosure, these enzymes may include those proteins which have the amino acid sequences of SEQ ID NOS described above as well as 80% or higher, 85% or higher, 90% or higher, 91% or higher, 92% or higher, 93% or higher, 94% or higher, 95% or higher, 96% or higher, 97% or higher, 98% or higher, 99% or higher homology or identity to the above amino acid sequences, as long as these proteins have a biological activity identical or corresponding to each of these enzymes.

Additionally, it is apparent that any protein which has an amino acid sequence with deletion, modification, substitution, or addition in part of the sequence can also be included within the scope of the present disclosure, as long as the amino acid sequence has a homology or identity to the SEQ ID NOS described above and has a biological activity substantially identical or corresponding to the enzyme proteins of the SEQ ID NOS described above.

As used herein, the term “homology or identity” refers to a degree of matching between given amino acid sequences or nucleotide sequences and may be expressed as a percentage. In the present disclosure, a homologous sequence having an activity identical or similar to a given amino acid sequence or nucleotide sequence is represented as “% homology” or “% identity”. For example, homology may be confirmed using standard software, specifically BLAST 2.0, for calculating parameters such as score, identity, and similarity or by comparing sequences by southern hybridization under defined stringent conditions. Defined appropriate hybridization conditions may be within the scope of the art and may be determined by a method well known to those skilled in the art (e.g., J. Sambrook et al., Molecular Cloning, A Laboratory Manual, 2nd Edition, Cold Spring Harbor Laboratory press, Cold Spring Harbor, N.Y., 1989; F. M. Ausubel et al., Current Protocols in Molecular Biology, John Wiley & Sons, Inc., New York). The term “stringent conditions” refers to conditions which enables specific hybridization between polynucleotides. For example, such conditions are specifically disclosed in a literature (e.g., J. Sambrook et al., supra).

The 2-dehydro-3-deoxyphosphoheptonate aldolase, phosphoenolpyruvate synthetase, and transketolase of the present disclosure may include the polynucleotides encoding proteins which have the amino acid sequences of the SEQ ID NOS described above, or 80% or higher, 85% or higher, 90% or higher, 91% or higher, 92% or higher, 93% or higher, 94% or higher, 95% or higher, 96% or higher, 97% or higher, 98% or higher, 99% or higher homology or identity to the amino acid sequences of the SEQ ID NOS described above, as long as these polynucleotides have a biological activity identical or corresponding to each of these enzymes.

Additionally, considering the codons preferred in an organism, where the protein is to be expressed, due to codon degeneracy, various modifications may be performed in the coding region of the nucleotide sequence within the scope not altering the amino acid sequence of the protein to be expressed from the coding region. Therefore, any polynucleotide having a sequence encoding each of the enzyme proteins can be included without limitation.

Additionally, any sequence which encodes a protein having an activity of the 2-dehydro-3-deoxyphosphoheptonate aldolase, phosphoenolpyruvate synthetase, or transketolase enzyme proteins by hybridizing with any probe that can be prepared from known gene sequences (e.g., complementary sequences to all or part of the above polynucleotide sequence) under stringent conditions, may be included without limitation.

The term “stringent conditions” refers to conditions which enables specific hybridization between polynucleotides. Such conditions are specifically described in a literature (e.g., J Sambrook et al., supra). For example, the condition for a hybridization between genes having a high homology or identity, a homology or identity of 40% or higher, specifically 90% or higher, more specifically 95% or higher, even more specifically 97% or higher, and most specifically 99% or higher, without a hybridization between genes having a homology or identity of lower than the homologies or identities described above; or conventional washing condition for southern hybridization, i.e., washing once, specifically, twice or three times at a salt concentration and temperature corresponding to 60° C., 1×SSC, 0.1% SDS, specifically 60° C., 0.1×SSC, 0.1% SDS, and more specifically 68° C., 0.1×SSC, 0.1% SDS, may be listed.

Hybridization requires that two polynucleotides have complementary sequences, although mismatches between bases are possible depending on the stringency of the hybridization. The term “complementary” is used to describe the relationship between nucleotide bases that can hybridize with each other. For example, with respect to DNA, adenosine is complementary to thymine, and cytosine is complementary to guanine. Accordingly, the present disclosure may also include isolated polynucleotide fragments complementary to the entire sequence as well as substantially similar polynucleotide sequences.

Specifically, polynucleotides having a homology or identity can be detected using hybridization conditions that include a step of hybridizing at a Tm value of 55° C. and using the conditions described above. Additionally, the Tm value may be 60° C., 63° C., or 65° C., but the temperature is not limited thereto and may be appropriately adjusted by those skilled in the art according to the purpose.

The stringency suitable for the hybridization of polynucleotides depends on the length and degree of complementarity of the polynucleotides, and the variables are well known in the art (see Sambrook et al., supra, 9.50 to 9.51 and 11.7 to 11.8). As used herein, the term “enhancement of activity” means that an activity of an enzyme protein is introduced, or the activity is enhanced compared to its endogenous activity or the activity before its modification in which a microorganism possesses. The “introduction” of an activity means that a microorganism naturally or artificially exhibits the activity of a particular protein which was not originally possessed in the microorganism. Specifically, a microorganism with an enhanced activity of an enzyme protein refers to a microorganism in which the activity of an enzyme protein is enhanced compared to that of a natural wild-type microorganism or non-modified microorganism. The enhancement of an activity may include, for example, both the enhancement of an activity by introducing an exogenous 2-dehydro-3-deoxyphosphoheptonate aldolase, phosphoenolpyruvate synthetase, and/or transketolase into a microorganism; or the enhancement of the activity of the endogenous 2-dehydro-3-deoxyphosphoheptonate aldolase, phosphoenolpyruvate synthetase, and/or transketolase.

Specifically, in the present disclosure, the method for the enhancement of an activity may include:
(1) a method of increasing the copy number of the polynucleotides encoding the enzymes;
(2) a method of modifying the expression control sequence for the increase of the expression of the polynucleotides;
(3) a method of modifying the polynucleotide sequences on the chromosome for the enhancement of the activities of the enzymes; or
(4) a method of modifying for the enhancement by a combination of the above methods (1) to (3), etc., but the methods are not limited thereto.

The method (1) of increasing the copy number of the polynucleotides may be performed in a form where the polynucleotide is operably linked to a vector or by inserting the polynucleotide into the chromosome of a host cell, but the method is not particularly limited thereto. Additionally, as an alternative, the copy number may be increased by introducing an exogenous polynucleotide exhibiting the activity of an enzyme or a codon-optimized modified polynucleotide of the polynucleotide above into a host cell. The exogenous polynucleotide may be used without limitation in its origin or sequence as long as the polynucleotide exhibits an activity identical or similar to the enzyme. The introduction may be performed by those skilled in the art by appropriately selecting a known transformation method, and the activity of the enzyme may be enhanced such that the introduced polynucleotide is expressed in the host cell thereby producing the enzyme.

Next, the method (2) of modifying the expression control sequence for the increase of the expression of the polynucleotides may be performed by inducing a modification in the sequence by deletion, insertion, non-conservative or conservative substitution, or a combination thereof so as to further enhance the activity of the expression control sequence; or by replacing the nucleic acid sequence with a nucleic acid sequence having a stronger activity, but the method is not particularly limited thereto. The expression control sequence may include a promoter, an operator sequence, a sequence encoding a ribosome-binding site, sequences controlling the termination of transcription and translation, etc., but the expression control sequence is not particularly limited thereto.

Specifically, a strong heterologous promoter instead of the original promoter may be linked upstream of the polynucleotide expression unit, and examples of the strong promoter may include a CJ7 promoter, a lysCP1 promoter, an EF-Tu promoter, a groEL promoter, an aceA or aceB promoter, etc. More specifically, the polynucleotide expression unit may be operably linked to a Corynebacterium-derived promoter such as lysCP1 promoter (WO 2009/096689), a CJ7 promoter (WO 2006/065095), an SPL promoter (KR 10-1783170 B), or an o2 promoter (KR 10-1632642 B) so as to improve the expression rate of the polynucleotide encoding the enzyme, but the method is not limited thereto.

Additionally, the method (3) of modifying the polynucleotide sequence on the chromosome may be performed by inducing a modification in the expression control sequence by deletion, insertion, non-conservative or conservative substitution, or a combination thereof so as to further enhance the activity of the polynucleotide sequence; or by replacing the nucleic acid sequence with an improved polynucleotide sequence having a stronger activity, but the method is not particularly limited thereto.

Lastly, the method (4) of modifying for enhancement by a combination of the methods (1) to (3) may be performed by applying one or more methods together among the methods: a method of increasing the copy number of the polynucleotides encoding the enzymes; a method of modifying the expression control sequence for the increase of the expression; and a method of modifying the polynucleotide sequences on the chromosome, or a method of modifying the exogenous polynucleotides exhibiting the activity of the enzyme or a codon-optimized modified polynucleotide thereof.

The polynucleotides may be described as genes when they are an assembly of polynucleotides capable of functioning. In the present disclosure, polynucleotides and genes can be used interchangeably, and polynucleotide sequences and nucleotide sequences can be used interchangeably.

As used herein, the term “vector” refers to a DNA construct including a nucleotide sequence of a polynucleotide encoding a target protein, in which the target protein is operably linked to a suitable control sequence so that it can be expressed in an appropriate host. The control sequence may include a promoter capable of initiating transcription, any operator sequence for controlling the transcription, a sequence encoding an appropriate mRNA ribosome-binding site, and sequences for controlling the termination of transcription and translation. The vector, after being transformed into a suitable host cell, may be replicated or function irrespective of the host genome, or may be integrated into the host genome itself.

The vector used in the present disclosure is not particularly limited as long as the vector can be replicated in a host cell, and any vector known in the art may be used. Examples of conventional vectors may include a natural or recombinant plasmid, cosmid, virus, and bacteriophage. For example, pWE15, M13, MBL3, MBL4, IXII, ASHII, APII, t10, t11, Charon4A, and Charon21A, etc. may be used as a phage vector or cosmid vector; and pBR, pUC, pBluescriptII, pGEM, pTZ, pCL, and pET, etc. may be used as a plasmid vector. Specifically, vectors such as pDZ, pACYC177, pACYC184, pCL, pECCG117, pUC19, pBR322, pMW118, pCC1BAC, pSKH, pRS-413, pRS-414, and pRS-415, etc. may be used, but the vectors are not limited thereto.

The vectors to be used in the present disclosure are not particularly limited, but any known expression vector may be used. Additionally, a polynucleotide encoding a target protein in the chromosome can be inserted into the chromosome with a vector for chromosomal insertion in a cell. The insertion of a polynucleotide into the chromosome can be performed using any method known in the art (e.g., by homologous recombination), but the method of insertion is not limited thereto. A selection marker for confirming the insertion into the chromosome may be further included. The selection marker is used for selection of cells transformed with a vector (i.e., to confirm whether a target nucleic acid molecule has been inserted) and markers capable of providing selectable phenotypes (e.g., drug resistance, auxotrophy, resistance to cytotoxic agents, and expression of surface proteins) may be used. Under the circumstances where selective agents are treated, only the cells capable of expressing the selection markers can survive or express other phenotypic traits, and thus the transformed cells can easily be selected.

As used herein, the term “transformation” refers to the introduction of a vector including a polynucleotide encoding a target protein into a host cell so that the protein encoded by the polynucleotide can be expressed in the host cell. The transformed polynucleotide may not be particularly limited as long as it can be expressed in a host cell, regardless of whether the transformed polynucleotide is inserted into the chromosome of the host cell to be located therein or located outside of the chromosome. Additionally, the polynucleotide includes DNA and RNA encoding a target protein. As long as the polynucleotide can be introduced into a host cell and expressed therein, it does not matter in which form the polynucleotide is introduced. For example, the polynucleotide may be introduced into a host cell in the form of an expression cassette, which is a gene construct including all essential elements for self-expression. The expression cassette may include a promoter, which is conventionally operably linked to the polynucleotide, a transcription termination signal, a ribosome-binding site, and a translation termination signal. The expression cassette may be a self-replicable expression vector. Additionally, the polynucleotide may be one which is introduced into a host cell as a polynucleotide itself and linked to a sequence necessary to be expressed in a host cell, but is not limited thereto. The methods for transformation include any method for introducing a nucleic acid into a cell, and may be performed by selecting a suitable standard technique known in the art depending on a host cell. For example, the transformation methods may include electroporation, calcium phosphate (CaPO₄) precipitation, calcium chloride (CaCl₂)) precipitation, microinjection, polyethylene glycol (PEG) method, EAE-dextran method, cationic liposome method, lithium acetate-DMSO method, etc., but the methods are not limited thereto.

Additionally, as used herein, the term “operably linked” means a functional linkage between a promoter sequence which initiates and mediates transcription of the polynucleotide encoding the target protein of the present disclosure and the polynucleotide sequence. An operable linkage may be prepared by a genetic recombination technique known in the art, and site-specific DNA cleavage and ligation may be prepared using a restriction enzyme, a ligase, etc., known in the art, without being limited thereto.

In the microorganism of the present disclosure, the activity of 3-dehydroquinate dehydratase may be further inactivated.

As used herein, the term “3-dehydroquinate dehydratase” refers to an enzyme that catalyzes the reversible reaction in the reaction scheme below, and specifically, it can convert 3-dehydroquinate to 3-dehydroshikimate, but is not limited thereto.

3-dehydroquinate↔3-dehydroshikimate+H₂O [Reaction Scheme]

3-dehydroquinate dehydratase is not limited to its origin or sequence because there are cases where the proteins showing the activity of 3-dehydroquinate dehydratase differ in their amino acid sequences depending on the species of a microorganism or the microorganism. Specifically, the 3-dehydroquinate dehydratase may be a protein including the amino acid sequence of SEQ ID NO: 90, but is not limited thereto. Additionally, the 3-dehydroquinate dehydratase may include an amino acid sequence of SEQ ID NO: 90 or an amino acid sequence having a homology or identity of at least 80%, 90%, 95%, 96%, 97%, 98%, or 99% to SEQ ID NO: 90. Additionally, it is apparent that any amino acid sequence with deletion, modification, substitution, or addition in part of the sequence can also be included within the scope of the present disclosure, as long as the amino acid sequence has the homology or identity described above and has a biological activity identical or corresponding to the above protein.

As used herein, the term “inactivation” refers to a case where the activity of an enzyme protein is weakened compared to the endogenous activity of the enzyme protein or its activity before modification originally possessed by a microorganism; a case where the protein is not expressed at all; or a case where the protein is expressed but has no activity. The inactivation is a concept which includes: a case where the activity of an enzyme itself is weakened compared to its endogenous activity possessed by a microorganism due to a modification of the polynucleotide encoding the enzyme, etc. or the activity is removed; a case where the degree of overall intracellular activity of an enzyme is lower compared to that of its wild-type microorganism or the activity is removed, due to the inhibition of expression or translation of the gene encoding the enzyme, etc.; a case where all or part of the gene encoding the enzyme is deleted; and a combination thereof, but the inactivation is not limited thereto. That is, a microorganism in which the activity of an enzyme is inactivated refers to a microorganism in which the activity of an enzyme protein is lower compared to that of its natural wild-type microorganism or non-modified microorganism or in which the activity is removed.

The inactivation of the activity of an enzyme may be achieved by the application of various methods known in the art. Examples of the above methods may include: 1) a method of deleting all or part of the gene encoding the enzyme on the chromosome; 2) a method of modifying the expression control sequence to reduce the expression of the gene encoding the protein on the chromosome; 3) a method of modifying the sequence of the gene encoding the protein on the chromosome so that the activity of the protein is removed or weakened; 4) a method of introducing an antisense oligonucleotide (e.g., antisense RNA), which binds complementarily to a transcript of the gene encoding the protein on the chromosome; 5) a method of making the attachment of a ribosome impossible by a secondary structure formed by adding a sequence, which is complementary to the Shine-Dalgarno (SD) sequence, on the front end of the SD sequence of the gene encoding the protein on the chromosome; 6) a method of reverse transcription engineering (RTE), in which a promoter that is transcribed reversely is added to the 3′ terminal of the open reading frame (ORF) of the polynucleotide sequence encoding the protein, etc.; and the inactivation may be achieved by a combination thereof, but the methods are not particularly limited thereto.

The method of deleting all or part of the gene encoding the enzyme on the chromosome may be performed by replacing the polynucleotide encoding the endogenous target protein within the chromosome with a polynucleotide or marker gene having a partially deleted nucleotide sequence using a vector for chromosomal insertion. As an example of the method for deleting all or part of a polynucleotide, a method for deleting a polynucleotide by homologous recombination may be used, but the method is not limited thereto.

The method of modifying the expression control sequence may be performed by inducing a modification of the nucleic acid sequence in the expression control sequence via deletion, insertion, conservative or non-conservative substitution, or a combination thereof so as to further weaken the activity of the expression control sequence; or by replacing the nucleic acid sequence with a nucleic acid sequence having a weaker activity. The expression control sequence may include a promoter, an operator sequence, a sequence encoding a ribosome-binding site, and a sequence for regulating transcription and translation, but is not limited thereto.

The method of modifying a gene sequence on the chromosome may be performed by inducing a modification in the gene sequence via deletion, insertion, conservative or non-conservative substitution, or a combination thereof so as to further weaken the activity of the enzyme; or by replacing the gene sequence with a gene sequence modified to have a weaker activity or a gene sequence modified to have no activity at all, but the method is not limited thereto.

In the above, the term “part”, although what it refers to may vary depending on the kinds of polynucleotide, may be specifically 1 nucleotide to 300 nucleotides, more specifically 1 nucleotide to 100 nucleotides, and even more specifically 1 nucleotide to 50 nucleotides, but is not particularly limited thereto.

In the microorganism of the present disclosure, the activity of 3-dehydroquinate synthase protein can be further strengthened compared to that of a non-modified microorganism.

The 3-dehydroquinate synthase refers to an enzyme that catalyzes the reversible reaction of the following reaction scheme, and specifically can synthesize 3-dehydroquinate (3-DHQ), but is not limited thereto.

3-deoxy-arabino-heptulosonate-7-phosphate↔3-dehydroquinate+phosphate [Reaction Scheme]

As used herein, the term “mycosporine-like amino acid (MAA)” refers to a cyclic compound that absorbs ultraviolet (UV) rays. In the present disclosure, the mycosporine-like amino acid is not limited as long as it can absorb UV rays, and specifically, it may be a compound which has the central ring of cyclohexanone or cyclohexenimine; or may be a compound in which various materials (e.g., amino acids, etc.) are bound to the central ring, and more specifically, it may be mycosporine-2-glycine, palythinol, palythenic acid, deoxygadusol, mycosporine-methylamine-threonine, mycosporine-glycine-valine, palythine, asterina-330, shinorine, porphyra-334, euhalothece-362, mycosporine-glycine, mycosporine-ornithine, mycosporine-lysine, mycosporine-glutamic acid-glycine, mycosporine-methylamine-serine, mycosporine-taurine, palythene, palythine-serine, palythine-serine-sulfate, palythinol, usujirene, or a combination thereof.

In the present disclosure, the term mycosporine-like amino acid may be used interchangeably with MAA and MAAs.

As used herein, the term “microorganism producing a mycosporine-like amino acid (MAA)” may refer to a microorganism which includes a gene of an enzyme involved in the biosynthesis of MAA or a cluster of these genes, or a microorganism in which the cluster is introduced or enhanced. Additionally, as used herein, the term “mycosporine-like amino acid (MAA) biosynthesis gene cluster” may refer to a group of genes encoding the enzymes involved in MAA biosynthesis, and specifically, may include an MAA biosynthesis gene, a gene of an enzyme having an activity of attaching an additional amino acid residue to MAA, or a cluster of the above genes. The MAA biosynthesis gene includes both the exogenous genes and/or endogenous genes of a microorganism, as long as the microorganism including such gene can produce MAA. The exogenous genes may be homogeneous or heterogeneous.

The species of the microorganisms from which the MAA biosynthesis gene is derived is not limited, as long as the microorganisms including the same can produce enzymes involved in MAA biosynthesis and consequently can produce MAA. Specifically, the species of the microorganisms from which the MAA biosynthesis gene is derived may be cyanobacteria (e.g., Anabaena variabilis, Nostoc punctiforme, Nodularia spumigena, Cyanothece sp. PCC 7424, Lyngbya sp. PCC 8106, Microcystis aeruginosa, Microcoleus chthonoplastes, Cyanothece sp. ATCC 51142, Crocosphaera watsonii, Cyanothece sp. CCY 0110, Cylindrospermum stagnale sp. PCC 7417, Aphanothece halophytica, or Trichodesmium erythraeum); fungi (e.g., Magnaporthe orzyae, Pyrenophora tritici-repentis, Aspergillus clavatus, Nectria haematococca, Aspergillus nidulans, Gibberella zeae, Verticillium albo-atrum, Botryotinia fuckeliana, or Phaeosphaeria nodorum); or Nematostella vectensis; Heterocapsa triquetra, Oxyrrhis marina, Karlodinium micrum, Actinosynnema mirum, etc., but are not limited thereto.

According to an embodiment, the microorganism of the present disclosure, which produces MAA, includes an MAA biosynthesis gene or a cluster thereof. Specifically, in the microorganism, a MAA biosynthesis gene cluster may be introduced or the activities of the proteins encoded by the genes may be enhanced compared to the endogenous activities or the activities before modification, but the microorganism is not limited thereto.

Additionally, although the MAA biosynthesis gene are not limited in their names of the enzymes or the microorganisms from which these genes are derived, as long as the microorganisms can produce MAA, the MAA biosynthesis gene may include gene encoding enzyme protein having an activity identical and/or similar to one or more, specifically, one or more, two or more, three or more, or all of the enzyme proteins selected from the group consisting of 2-demethyl 4-deoxygadusol synthase, O-methyltransferase, and a C—N ligase.

For example, the 2-demethyl 4-deoxygadusol synthase is an enzyme which converts sedoheptulose-7-phosphate to 2-demethyl-4-deoxygadusol. The O-methyltransferase is an enzyme which converts 2-demethyl-4-deoxygadusol to 4-deoxygadusol, and glycylation of the 4-deoxygadusol is catalyzed by the C—N ligase.

Additionally, the microorganism producing MAAs may include a gene of an enzyme, which has an activity of attaching an additional amino acid residue to MAA, or a cluster of the genes. Although the above gene or a cluster of the genes are not limited in their names of the enzymes or the microorganisms from which these genes are derived, as long as the microorganisms producing MAAs can produce MAAs to which two or more amino acid residues are attached. The microorganisms producing MAAs may include gene encoding enzyme protein having an activity identical and/or similar to one or more, specifically, one or more, two or more, three or more, or all of the enzyme proteins, selected from the group consisting of non-ribosomal peptide synthetase (NRPS), a non-ribosomal peptide synthetase-like enzyme (NRPS-like enzyme), and D-alanine D-alanine ligase (D-Ala D-Ala ligase; DDL).

Some of the MAAs include a second amino acid residue in a mycosporine-glycine. The one or more enzymes, which are selected from the group consisting of non-ribosomal peptide synthetase, a non-ribosomal peptide synthetase-like enzyme, and D-Ala D-Ala ligase, can attach a second amino acid residue to a mycosporine-glycine.

According to an embodiment, the microorganism producing MAAs may include, without limitation on enzyme names or the species of the microorganisms from which the MAA biosynthesis genes are derived, enzymes as long as they have an activity capable of attaching a second amino acid to a mycosporine-glycine, as in the non-ribosomal peptide synthetase, non-ribosomal peptide synthetase-like enzyme, and D-Ala D-Ala ligase.

For example, the non-ribosomal peptide synthetase-like enzyme (Ava_3855) in Anabaena variabilis or D-Ala D-Ala ligase (NpF5597) in Nostoc punctiforme can attach a serine residue to mycosporine-glycine to form shinorine. In another example, mycosporine-2-glycine can be formed by the attachment of a second glycine residue by D-Ala D-Ala ligase homolog (Ap_3855) in Aphanothece halophytica. Similarly, in Actinosynnema mirum, serine or alanine can be attached by D-Ala D-Ala ligase and thereby shinorine or mycosporine-glycine-alanine can be formed. The microorganism according to an embodiment of the present disclosure can select and include those enzymes which are suitable for the production of desired MAAs among the above-described enzymes or the enzymes having activities identical and/or similar to the above-described enzymes.

The 2-demethyl 4-deoxygadusol synthase, O-methyltransferase, a C—N ligase, non-ribosomal peptide synthetase, non-ribosomal peptide synthetase-like enzyme, and/or D-Ala D-Ala ligase that can be used in the present disclosure are not limited to the species of microorganisms from which these enzymes are derived, as long as these enzymes are known to be able to perform the functions and roles identical and/or similar to those of the above-described enzymes, and the range of homology or identity values among them is also not limited. For example, MylA, MylB, MylD, MylE, and MylC of C. stagnale PCC 7417 are homologous with the 2-demethyl 4-deoxygadusol synthase, O-methyltransferase, a C—N ligase, and a D-Ala D-Ala ligase derived from Anabaena variabilis and Nostoc punctiforme, and the degree of similarity between them is in a range of about 61% to about 88% (Appl Environ Microbiol, 2016, 82(20), 6167-6173; J Bacteriol, 2011, 193(21), 5923-5928). That is, the enzymes that can be used in the present disclosure are not significantly limited in the species from which the enzymes are derived, sequence homology, or sequence identity, as long as they are known to exhibit identical and/or similar functions and effects. Additionally, the non-patent documents described in Prior Art Documents are included in their entirety as a reference to the present disclosure as a whole.

Additionally, the MAA biosynthesis gene may be a polynucleotide encoding a protein which includes an amino acid sequence of SEQ ID NO: 115, 116, 117, 118, 119, 120, 121, or 122, but the MAA biosynthesis gene is not limited thereto.

Additionally, the MAA biosynthesis gene may include a nucleotide sequence encoding a protein which includes the amino acid sequence having 50%, 60%, or 70% or higher, specifically 80% or higher, more specifically 90% or higher, even more specifically 95% or higher, and most specifically 99% or higher homology or identity to the amino acid sequence of SEQ ID NO: 115, 116, 117, 118, 119, 120, 121, or 122, and may include without limitation a nucleotide sequence encoding a protein which is out of the range of the above homology or identity, as long as the microorganism can produce MMA. Specifically, the MAA biosynthesis gene may include a nucleotide sequence of SEQ ID NO: 102, 103, 104, 105, 106, 107, 108, or 109, but is not limited thereto.

Additionally, it is apparent that any amino acid sequence with deletion, modification, substitution, or addition in part of the sequence can also be included in the present disclosure, as long as the amino acid sequence has a homology or identity to the above sequences and substantially has a biological activity identical or corresponding to the proteins of the SEQ ID NOS described above.

Additionally, considering the codons preferred in an organism, where the protein is to be expressed, due to codon degeneracy, various modifications may be performed in the coding region of the nucleotide sequence within the scope not altering the amino acid sequence of the protein to be expressed from the coding region. Therefore, with regard to the MAA biosynthesis gene, any nucleotide sequence can be included in the present disclosure without limitation as long as the nucleotide sequence is a nucleotide sequence, which encodes a protein involved in the MAA biosynthesis.

Alternatively, any sequence which encodes a protein involved in the MAA biosynthesis, by hybridizing with any probe that can be prepared from known gene sequences (e.g., complementary sequences to all or part of the polynucleotide sequence) under stringent conditions, can be included in the present disclosure without limitation.

According to an embodiment, a microorganism producing MAA may include MAA biosynthesis genes having different origins.

In the present disclosure, the enhancement of a protein activity and/or the introduction of genes may be performed in a simultaneous, sequential, and reverse order regardless of the order.

The microorganism producing MAA can produce MAA by including a MAA biosynthesis gene cluster, and additionally, may be a microorganism in which the MAA-producing ability is increased by enhancing the activity of one or more proteins selected from the group consisting of 2-dehydro-3-deoxyphosphoheptonate aldolase, phosphoenolpyruvate synthetase, and transketolase. Additionally, the microorganism of the present disclosure is not limited as long as it is a microorganism in which the MAA-producing ability is increased by enhancing the activity of one or more proteins selected from the group consisting of 2-dehydro-3-deoxyphosphoheptonate aldolase, phosphoenolpyruvate synthetase. Specifically, the microorganism of the present disclosure may be a microorganism of the genus Corynebacterium, a microorganism of the genus Escherichia, or a yeast.

The microorganism of the genus Corynebacterium may be, specifically, Corynebacterium glutamicum, Corynebacterium ammoniagenes, Brevibacterium lactofermentum, Brevibacterium flavum, Corynebacterium thermoaminogenes, Corynebacterium efficiens, etc., and more specifically may be Corynebacterium glutamicum, but the microorganism is not limited thereto.

The microorganism of the genus Escherichia may be, specifically, Escherichia albertii, Escherichia coli, Escherichia fergusonii, Escherichia hermannii, Escherichia vulneris, etc., and more specifically may be Escherichia coli, but the microorganism is not limited thereto.

The yeast may be, specifically, Saccharomycotina and Taphrinomycotina of the phylum Ascomycota, Agaricomycotina of the phylum Basidiomycota, a microorganism belonging to the phylum Pucciniomycotina, etc., more specifically a microorganism of the genus Saccharomyces, a microorganism of the genus Schizosaccharomyces, a microorganism of the genus Phaffia, a microorganism of the genus Kluyveromyces, a microorganism of the genus Pichia, and a microorganism of the genus Candida, and more specifically Saccharomyces cerevisiae, but the microorganism is not limited thereto.

Still another aspect of the present disclosure provides a method for producing a mycosporine-like amino acid, which includes culturing the microorganism of the present disclosure in a medium; and recovering the mycosporine-like amino acid (MAA) from the cultured microorganism or medium.

The “microorganism” and “mycosporine-like amino acid (MAA)” are as described above.

As used herein, the term “culture” means that the microorganism is grown under appropriately controlled environmental conditions. The culture process of the present disclosure can be performed in a suitable culture medium and culture conditions known in the art. Such a culture process may be easily adjusted for use by those skilled in the art according to the microorganism to be selected. The step of culturing the microorganism may be performed in batch culture, continuous culture, fed-batch culture, etc. known in the art, but the step of culturing the microorganism is not particularly limited thereto. The medium and other culture conditions used for culturing the microorganism of the present disclosure are not particularly limited, but any medium used in the conventional culture for a microorganism may be used. Specifically, the microorganism of the present disclosure may be cultured under aerobic conditions in a conventional medium containing an appropriate carbon source, nitrogen source, phosphorus source, inorganic compound, amino acid, and/or vitamin, etc. while adjusting temperature, pH, etc. Specifically, the pH may be adjusted using a basic compound (e.g., sodium hydroxide, potassium hydroxide, or ammonia) or an acidic compound (e.g., phosphoric acid or sulfuric acid) so as to obtain an optimal pH (e.g., pH 5 to 9, specifically pH 6 to 8, and most specifically pH 6.8), but the method of pH adjustment is not limited thereto. Additionally, oxygen or oxygen-containing gas may be injected into the culture in order to maintain an aerobic state of the culture; or nitrogen, hydrogen, or carbon dioxide gas may be injected into the culture without gas injection in order to maintain an anaerobic or microaerobic state of the cultured, but the gas is not limited thereto. Additionally, the culture temperature may be maintained at 20° C. to 45° C., and specifically 25° C. to 40° C., and the culture may be performed for about 10 hours to about 160 hours, without being limited thereto. Additionally, during the culture, an antifoaming agent (e.g., fatty acid polyglycol ester) may be added to prevent foam generation, but is not limited thereto.

Additionally, as a carbon source to be used in the medium for culture, saccharides and carbohydrates (e.g., glucose, sucrose, lactose, fructose, maltose, molasses, starch, and cellulose), oils and fats (e.g., soybean oil, sunflower oil, peanut oil, and coconut oil), fatty acids (e.g., palmitic acid, stearic acid, and linoleic acid), alcohols (e.g., glycerol and ethanol), organic acids (e.g., acetic acid), etc. may be used alone or in combination, but the carbon source is not limited thereto. As a nitrogen source, a nitrogen-containing organic compound (e.g., peptone, yeast extract, meat gravy, malt extract, corn steep liquor, bean flour, and urea), and an inorganic compound (e.g., ammonium sulfate, ammonium chloride, ammonium phosphate, ammonium carbonate, and ammonium nitrate), etc. may be used alone or in combination, but the nitrogen source is not limited thereto. As a phosphorous source, potassium dihydrogen phosphate, dipotassium hydrogenphosphate, and sodium-containing salts corresponding thereto may be used alone or in combination, but the phosphorous source is not limited thereto. Additionally, the medium may include essential growth-promoting materials, such as a metal salt (e.g., magnesium sulfate or iron sulfate), amino acids, and vitamins.

The MAAs produced by culturing may be secreted into the medium or remain in the cells.

As used herein, the term “medium” refers to a culture medium for culturing the microorganism of the present disclosure and/or a product obtained after culture. The medium is a concept, which includes both a form where the microorganism is included and a form where the microorganism is removed from the microorganism-containing cultured solution by centrifugation, filtration, etc.

In the step of recovering the MAAs produced in the culturing step of the present disclosure above, the desired MAAs can be collected from the culture solution using an appropriate method known in the art according to the culture method. For example, centrifugation, filtration, anion-exchange chromatography, crystallization, HPLC, etc. can be used, and the desired MAAs can be recovered from the cultured microorganism or medium using an appropriate method known in the art. The step of recovering the MAAs may further include a separation step and/or a purification step.

Still another aspect of the present disclosure provides a use of the microorganism of the present disclosure for the production of a mycosporine-like amino acid (MMA).

The “microorganism” and “mycosporine-like amino acid” are as described above.

### MODE FOR CARRYING OUT THE INVENTION

Hereinafter, the present disclosure will be described in more detail with reference to the following Examples. However, these Examples are for illustrative purposes only and the scope of the disclosure is not limited by these Examples.

<Preparation of E. coli-Based Recombinant Microorganism Producing MAAs and Production of MAAs Using the Same>

### Example 1: Preparation of Strain in which Activity of 2-dehydro-3-deoxyphosphoheptonate Aldolase is Enhanced

To increase the MAAs-producing ability of a microorganism, E. coli strains in which the activity of 2-dehydro-3-deoxyphosphoheptonate aldolase is enhanced were prepared. Specifically, the aroG gene (2-dehydro-3-deoxyphosphoheptonate aldolase; SEQ ID NOS: 1 and 2) was further introduced based on an E. coli W3110 strain. The templates and primers used for the preparation of plasmids are shown in Table 1 below.

TABLE 1

| | | Primers Used |
|---|---|---|
| | | (Forward Direction, |
| PCR Product | Template Used | Reverse Direction) |
| fhuA arm 1 | W3110 genomic DNA | SEQ ID NO: 3, |
| | | SEQ ID NO: 4 |
| fhuA arm 2 | W3110 genomic DNA | SEQ ID NO: 5, |
| | | SEQ ID NO: 6 |
| Pn_aroG | W3110 genomic DNA | SEQ ID NO: 7, |
| | | SEQ ID NO: 8 |
| Ptrc | pECCG117 Ptrc_GFP | SEQ ID NO: 9, |
| | | SEQ ID NO: 10 |
| Pcj1 | pECCG117 Pcj1_GFP | SEQ ID NO: 11, |
| (KR10-620092B) | | SEQ ID NO: 12 |
| aroG | W3110 genomic DNA | SEQ ID NO: 13, |
| | | SEQ ID NO: 8 |

After amplifying gene fragments through PCR using the templates and primers above, the amplified fragments were ligated to a pSKH vector in which the fragments of fhuA arm 1 and fhuA arm 2 genes were treated with BamH1-SpeI restriction enzymes, using the In-FusionR HD cloning kit (Clontech), and the prepared vector was named as pSKH-ΔfhuA. Due to the deletion of the fhuA gene, the phage infection of E. coli is inhibited.

A Pn_aroG gene fragment was ligated to pSKH-ΔfhuA vector, which was cut with Spe1-EcoRV restriction enzymes, using the In-Fusion^{R} HD cloning kit (Clontech). After digesting Ptrc and Pcj1 gene fragments, which are known as enhanced promoters, with Spe1-Nde1 restriction enzymes and digesting an aroG gene fragment with Nde1-EcoRV restriction enzymes, the Ptrc and aroG gene fragments or the Pcj1 (Korean Patent No. 10-620092) and aroG gene fragments were respectively ligated to the pSKH-ΔfhuA vector which was cut with Spe1-EcoRV restriction enzymes, using the In-Fusion^{R} HD cloning kit (Clontech). The prepared vectors were named as pSKH-ΔfhuA-Pn-aroG, pSKH-ΔfhuA-Ptrc-aroG, and pSKH-ΔfhuA-Pcj1-aroG, respectively.

The above vectors were confirmed by sequencing with regard to the success of cloning and gene sequence in each vector, and then, were transformed into each wild-type E. coli W3110 strain by electroporation. Each transformed gene was introduced into the chromosome by primary recombination (cross-over), and the plasmid region was excised from the chromosome through secondary recombination (cross-over). For each of the transformed E. coli strains, in which the secondary recombination is completed, the introduction of the aroG gene was confirmed by PCR using primers of SEQ ID NOS: 14 (forward direction) and 8 (reverse direction).

### Example 2: Preparation of Vector Overexpressing Microalgae-Derived Shinorine Biosynthesis Gene

A. variabilis-based gene cluster for shinorine biosynthesis consists of four genes (Ava_ABCD), which encode 2-demethyl 4-deoxygadusol synthase, O-methyltransferase, a C—N ligase, and non-ribosomal peptide synthetase. The gene cluster for shinorine biosynthesis was identified using the genomic DNA of A. variabilis ATCC29413. A vector including a shinorine biosynthesis gene derived from A. variabilis ATCC29413 was prepared using the pECCG117_Pcj1_GFP_terminator vector. The name of the shinorine biosynthesis gene expression vector and the respective template and primers for the preparation of the vector are shown in Table 2 below.

TABLE 2

| | | Primers Used |
|---|---|---|
| | | (Forward Direction, |
| PCR Product | Template Used | Reverse Direction) |
| Ava_ABCD | genomic DNA of | SEQ ID NO: 15, |
| | A. variabilis ATCC29413 | SEQ ID NO: 16 |

Gene fragments were obtained using the template and primers above, and each gene fragment was ligated to a pECCG 117_Pcj1_GFP_terminator vector, which was treated with EcoRV-XbaI restriction enzymes, using the In-Fusion^{R} HD cloning kit (Clontech). The prepared vector was named as pECCG117 Pcj1_Ava_ABCD, and the success of cloning and the gene sequence of the vector were confirmed by sequencing. The nucleotide sequence of the Ava_ABCD gene is shown in SEQ ID NO: 17.

### Example 3: Evaluation of Shinorine-Producing Ability of Strain in which Activity of 2-dehydro-3-deoxyphosphoheptonate Aldolase is Enhanced

The pECCG117_Pcj1_Ava_ABCD plasmid prepared in Example 2 was introduced into each of the strains prepared in Example 1, in which an aroG gene is enhanced, and a wild-type W3110 strain by electroporation, and each transformed strain was plated on a LB solid medium. The strains were cultured in a 37° C. incubator overnight, and then one platinum loop thereof was inoculated into a 25 mL titration medium [medium composition: glucose 40 g/L, KH₂PO₄ 0.3 g/L, K2HPO₄ 0.6 g/L, (NH4)₂SO₄ 15 g/L, MgSO₄.7H₂O 1 g/L, NaCl 2.5 g/L, sodium citrate 1.2 g/L, yeast extract 2.5 g/L, calcium carbonate 40 g/L: pH 7.0], which was incubated in a 37° C. incubator at 200 rpm for 48 hours. The resulting strains were analyzed by HPLC (Waters Corp.) and the results are shown in Table 3 below.

TABLE 3

| | | Shinorine |
|---|---|---|
| | OD | Conc. |
| Name of Strain | (600 nm) | (mg/L) |
| W3110/pECCG117_Pcj1_Ava_ABCD | 19.4 | 348 |
| W3110ΔfhuA::Pn-aroG/ | 19.6 | 418 |
| pECCG117_Pcj1_Ava_ABCD | | |
| W3110ΔfhuA::Ptrc-aroG/ | 20.3 | 589 |
| pECCG117_Pcj1_Ava_ABCD | | |
| W3110ΔfhuA::Pcj1-aroG/ | 20.5 | 674 |
| pECCG117_Pcj1_Ava_ABCD | | |

As shown in Table 3 above, the concentration of shinorine produced in the strain (W3110ΔfhuA::Pn-aroG/pECCG117_Pcj1_Ava_ABCD), where the aroG gene is enhanced, was increased by about 20% compared to that of the control group. In particular, in the case of the strains where the aroG gene is enhanced by enhancing the promoter (i.e., W3110ΔfhuA::Ptrc-aroG/pECCG117_Pcj1_Ava_ABCD and W3110ΔfhuA::Pcj1-aroG/pECCG117_Pcj1_Ava_ABCD), the shinorine concentration was increased by 69% and 94%, respectively.

### Example 4: Preparation of Strain in which Activity of Phosphoenolpyruvate Synthetase is Enhanced

To increase the MAAs-producing ability of a microorganism, E. coli strains in which the activity of phosphoenolpyruvate synthetase is enhanced were prepared. Specifically, the pps gene (phosphoenolpyruvate synthetase; SEQ ID NOS: 18 and 19) was further introduced based on an E. coli W3110 strain. The template and primers used for the preparation of the plasmid are shown in Table 4 below.

TABLE 4

| | | Primers Used |
|---|---|---|
| | | (Forward Direction, |
| PCR Product | Template Used | Reverse Direction) |
| Pn-pps | W3110 genomic DNA | SEQ ID NO: 20, |
| | | SEQ ID NO: 21 |
| pps | W3110 genomic DNA | SEQ ID NO: 22, |
| | | SEQ ID NO: 21 |

After amplifying gene fragments using the template and primers above, the Pn_pps gene fragment was ligated to pSKH-ΔfhuA vector, which was cut with Spe1-EcoRV restriction enzymes, using the In-Fusion^{R} HD cloning kit (Clontech). After digesting the Ptrc and Pcj1 gene fragments prepared in Example 1 with Spe1-Nde1 restriction enzymes and digesting the pps gene fragment with Nde1-EcoRV restriction enzymes, the Ptrc and pps gene fragments or the Pcj1 and pps gene fragments were respectively ligated to the pSKH-ΔfhuA vector, which was cut with Spe1-EcoRV restriction enzymes, using the In-Fusion^{R} HD cloning kit (Clontech). The prepared vectors were named as pSKH-ΔfhuA-Pn-pps, pSKH-ΔfhuA-Ptrc-pps, and pSKH-ΔfhuA-Pcj1-pps, respectively.

The above vectors were confirmed by sequencing with regard to the success of cloning and gene sequence in each vector, and then, transformed into each wild-type E. coli W3110 strain by electroporation. Each transformed gene was introduced into the chromosome by primary recombination (cross-over), and the plasmid region was excised from the chromosome through secondary recombination (cross-over). For each of the transformed E. coli strains, in which the secondary recombination is completed, the introduction of the pps gene was confirmed by PCR using primers of SEQ ID NOS: 14 (forward direction) and 21 (reverse direction).

### Example 5: Evaluation of Shinorine-Producing Ability of Strain in which Activity of Phosphoenolpyruvate Synthetase is Enhanced

The pECCG117_Pcj1_Ava_ABCD plasmid prepared in Example 2 was introduced into each of the strains prepared in Example 4, in which a pps gene is introduced, and a wild-type W3110 strain by electroporation, and each transformed strain was plated on a LB solid medium. The strains were cultured in a 37° C. incubator overnight, and one platinum loop thereof was inoculated into the 25 mL titration medium of Example 3, which was then incubated in a 37° C. incubator at 200 rpm for 48 hours. The results are shown in Table 5 below.

TABLE 5

| | | Shinorine |
|---|---|---|
| | OD | Conc. |
| Name of Strain | (600 nm) | (mg/L) |
| W3110/pECCG117_Pcj1Ava_ABCD | 19.4 | 348 |
| W3110ΔfhuA::Pn-pps/ | 21.3 | 494 |
| pECCG117_Pcj1_Ava_ABCD | | |
| W3110ΔfhuA::Ptrc-pps/ | 20.8 | 511 |
| pECCG117_Pcj1_Ava_ABCD | | |
| W3110ΔfhuA::Pcj1-pps/ | 21.4 | 556 |
| pECCG117_Pcj1_Ava_ABCD | | |

As shown in Table 5 above, the concentration of shinorine produced in the strain where a pps gene is enhanced was increased by 41%, and in the case where its activity is enhanced by replacing with a strong promoter, the shinorine concentration was increased up to 60% compared to that of the control group.

### Example 6: Preparation of Strain in which Activity of Transketolase I/II is Enhanced

To increase the MAAs-producing ability of a microorganism, E. coli strains in which the activity of transketolase is enhanced were prepared. Specifically, based on an E. coli W3110 strain, a tktA gene (transketolase; SEQ ID NOS: 23 and 24) was introduced thereinto. The template and primers used in the preparation of plasmids are shown in Table 6 below.

TABLE 6

| | | Primers Used |
|---|---|---|
| | | (Forward Direction, |
| PCR Product | Template Used | Reverse Direction) |
| Pn-tkt4 | W3110 genomic DNA | SEQ ID NO: 25, |
| | | SEQ ID NO: 26 |
| tktA | W3110 genomic DNA | SEQ ID NO: 27, |
| | | SEQ ID NO: 26 |

After amplifying gene fragments through PCR using the template and primers above, a Pn_tktA gene fragment was ligated to pSKH-ΔfhuA vector, which was cut with Spe1-EcoRV restriction enzymes, using the In-Fusion^{R} HD cloning kit (Clontech). After digesting the Ptrc and Pcj1 gene fragments prepared in Example 1 with Spe1, Nde1 restriction enzymes and digesting a tktA gene fragment with Nde1-EcoRV restriction enzymes, the Ptrc and tktA gene fragments or the Pcj1 and tktA gene fragments were respectively ligated to pSKH-ΔfhuA vector, which was cut with Spe1-EcoRV restriction enzymes, using the In-Fusion^{R} HD cloning kit (Clontech). The prepared vectors were named as pSKH-ΔfhuA-Pn-tktA, pSKH-ΔfhuA-Ptrc-tktA, and pSKH-ΔfhuA-Pcj1-tktA, respectively.

The above vectors were confirmed by sequencing with regard to the success of cloning and gene sequence in each vector, and then, transformed into each wild-type E. coli W3110 strain by electroporation. Each transformed gene was introduced into the chromosome by primary recombination (cross-over), and the plasmid region was excised from the chromosome through secondary recombination (cross-over). For each of the transformed E. coli strains, in which the secondary recombination is completed, the introduction of the tktA gene was confirmed by PCR using primers of SEQ ID NOS: 14 (forward direction) and 26 (reverse direction).

### Example 7: Evaluation of Shinorine-Producing Ability in Strain in which Activity of Transketolase is Enhanced

The pECCG117_Pcj1_Ava_ABCD plasmid prepared in Example 2 was introduced into each of the strains prepared in Example 6, in which a tktA gene is introduced, and a wild-type W3110 strain by electroporation, and each transformed strain was plated on a LB solid medium. The strains were cultured in a 37° C. incubator overnight, and one platinum loop of the overnight culture of each strain was inoculated into the 25 mL titration medium of Example 3, which was then incubated in a 37° C. incubator at 200 rpm for 48 hours. The results are shown in Table 7 below.

TABLE 7

| | | Shinorine |
|---|---|---|
| | OD | Conc. |
| Name of Strain | (600 nm) | (mg/L) |
| W3110/pECCG117_Pcj1_Ava_ABCD | 19.4 | 348 |
| W3110ΔfhuA::Pn-tkt4/ | 19.5 | 364 |
| pECCG117_Pcj1_Ava_ABCD | | |
| W3110ΔfhuA::Ptrc-tktA/ | 19.3 | 447 |
| pECCG117_Pcj1_Ava_ABCD | | |
| W3110ΔfhuA::Pcj1-tktA/ | 19.5 | 461 |
| pECCG117_Pcj1_Ava_ABCD | | |

As shown in Table 7 above, the concentration of shinorine produced in the strain where the tktA gene is enhanced was increased by 4.5%, and in the case where its activity is enhanced by replacing with a strong promoter, the shinorine concentration was increased up to 32% compared to that of the control group.

### Example 8: Preparation of Strain in which Activities of 2-Dehydro-3-Deoxyphosphoheptonate Aldolase/Phosphoenolpyruvate Synthetase/Transketolase are Enhanced

To increase the MAAs-producing ability of a microorganism, E. coli strains in which the activity of each of 2-dehydro-3-deoxyphosphoheptonate aldolase/phosphoenolpyruvate synthetase/transketolase is enhanced were prepared. Specifically, based on an E. coli W3110 strain, each of an aroG gene, a pps gene, and a tktA gene was further introduced thereinto. The templates and primers used in the preparation of plasmids are shown in Table 8 below.

TABLE 8

| | | Primers Used |
|---|---|---|
| | | (Forward Direction, |
| PCR Product | Template Used | Reverse Direction) |
| Pcj1-aroG | pSKH-ΔfhuA-Pcj1-aroG | SEQ ID NO: 11, |
| | | SEQ ID NO: 28 |
| Pcj1-pps | pSKH-ΔfhuA-Pcj1-pps | SEQ ID NO: 29, |
| | | SEQ ID NO: 30 |
| Pcj1-tktA | pSKH-ΔfhuA-Pcj1-tktA | SEQ ID NO: 31, |
| | | SEQ ID NO: 26 |

After amplifying gene fragments using the templates and primers above, each gene fragment thereof was ligated to the pSKH-ΔfhuA vector, which was cut with Spe1-EcoRV restriction enzymes, using the In-Fusion^{R} HD cloning kit (Clontech). The prepared vector was named as pSKH-ΔfhuA-Pcj1-aroG-Pcj1-ppsA-Pcj1-tktA.

The above vector was confirmed by sequencing with regard to the success of cloning and gene sequences in the vector, and then, transformed into a wild-type E. coli W3110 strain by electroporation. The transformed genes were introduced into the chromosome by primary recombination (cross-over), and the plasmid region was excised from the chromosome through secondary recombination (cross-over). For the transformed E. coli strains, in which the secondary recombination is completed, the introduction of the aroG, pps, and tktA genes was confirmed by PCR using primers of SEQ ID NOS: 14 (forward direction) and 26 (reverse direction).

### Example 9: Evaluation of Shinorine-Producing Ability in Strain in which Activities of 2-Dehydro-3-Deoxyphosphoheptonate Aldolase, Phosphoenolpyruvate Synthetase, and Transketolase are Enhanced

The pECCG117_Pcj1_Ava_ABCD plasmid prepared in Example 2 was introduced into each of the strain prepared in Example 8, in which aroG, pps, and tktA genes are introduced, and a wild-type W3110 strain by electroporation, and each transformed strain was plated on a LB solid medium. The strains were cultured in a 37° C. incubator overnight, and one platinum loop of the overnight culture of each strain was inoculated into the 25 mL titration medium of Example 3, which was then incubated in a 37° C. incubator at 200 rpm for 48 hours. The results are shown in Table 9 below.

TABLE 9

| | | Shinorine |
|---|---|---|
| | OD | Conc. |
| Name of Strain | (600 nm) | (mg/L) |
| W3110/pECCG117_Pcj1_Ava_ABCD | 19.4 | 348 |
| W3110ΔfhuA::Pcj1- | 18.2 | 1,279 |
| aroG-Pcj1-pps-Pcj1-tktA/ | | |
| pECCG117_Pcj1_Ava_ABCD | | |

As shown in Table 9 above, the concentration of shinorine produced in the strain where the three kinds of genes (i.e., aroG, pps, and tktA) are combined and enhanced was increased by 267% compared to that of the control group. This is an unexpected result showing an improvement beyond expectation compared to the sum of the maximum increases obtained by replacing the promoter of each gene with a strong promoter. That is, it was confirmed that when the three genes (i.e., aroG, pps, and tktA) are combined, it is possible to produce shinorine at a higher concentration.

### Example 10: Preparation of Strain in which Activity of 3-Dehydroquinate Dehydratase is Inactivated

To increase the MAAs-producing ability of a microorganism, E. coli strains in which the activity of 3-dehydroquinate dehydratase (aroD) is inactivated were prepared.

Specifically, a chloramphenicol resistance gene of a pKD3 plasmid was used as a gene insertion marker, and an aroD-deletion cassette, which includes part of an aroD gene and the chloramphenicol resistance gene of a pKD3 plasmid, was prepared by PCR using the primers of SEQ ID NO: 32 (forward direction) and 33 (reverse direction). Competent cells were prepared by transforming a wild-type E. coli W3110 strain and the strain prepared in Example 8, in which the aroG, pps, and tktA genes are introduced, with a pKD46 plasmid including a lambda red recombinase gene, followed by inducing the expression of the corresponding gene using arabinose. After introducing the aroD-deletion cassette into the competent cells by electroporation, the resulting competent cells were plated on a LB solid medium containing 30 mg/L of chloramphenicol. The thus-obtained strain was subjected to PCR using the primers of SEQ ID NOS: 34 (forward direction) and 35 (reverse direction), and the aroD gene deletion was confirmed by observing the 1,300 bp amplified fragment.

### Example 11: Evaluation of Shinorine-Producing Ability of Strain in which 3-Dehydroquinate Dehydratase is Inactivated

The pECCG117 Pcj1_Ava_ABCD plasmid prepared in Example 2 was introduced into the strain prepared in Example 10, in which an aroD gene is deleted, by electroporation, and the resulting strain was plated on a LB solid medium. The strain was cultured in a 37° C. incubator overnight, and one platinum loop thereof was inoculated into the 25 mL titration medium of Example 3, which was incubated in a 37° C. incubator at 200 rpm for 48 hours. The results are shown in Table 10 below.

TABLE 10

| | | Shinorine |
|---|---|---|
| | OD | Conc. |
| Name of Strain | (600 nm) | (mg/L) |
| W3110ΔfhuA::Pcj1-aroG- | 18.6 | 1,248 |
| Pcj1-ppsA-Pcj1-tktA/ | | |
| pECCG117_PCJ1_Ava_ABCD | | |
| W3110ΔaroDΔfhuA::Pcj1- | 17.3 | 2,077 |
| aroG-Pcj1-ppsA-Pcj1-tktA/ | | |
| pECCG117_PCJ1_Ava_ABCD | | |

As shown in Table 10 above, the concentration of shinorine produced in the strain, where the aroD gene is further deleted, was increased by 66% compared to that of the strain producing shinorine where the aroG, pps, and tktA genes are enhanced. The W3110ΔfhuA::Pcj1-aroG-Pcj1-ppsA-Pcj1-tktA/pECCG117_PCJ1_Ava_ABCD strain, which is a strain in which the aroG, pps, and tktA genes are enhanced, was named as CB06-0020, and deposited under the Budapest Treaty on Feb. 14, 2018, in the Korean Culture Center of Microorganisms (KCCM) and assigned Accession No. KCCM12224P.

<Preparation of Corynebacterium glutamicum-Based Recombinant Microorganism Producing MAAs and Production of MAAs Using the Same>

### Example 12: Preparation of Vector in which Activity of 2-Dehydro-3-Deoxyphosphoheptonate Aldolase is Enhanced and Evaluation of Shinorine-Producing Ability of the Same

To increase the MAAs-producing ability of a microorganism, E. coli strains in which the activity of 2-dehydro-3-deoxyphosphoheptonate aldolase is enhanced were prepared. Specifically, the aroG gene (2-dehydro-3-deoxyphosphoheptonate aldolase; SEQ ID NOS: 36 and 37) was further introduced based on a Corynebacterium glutamicum ATCC13032 strain. The template and primers used for the preparation of the plasmid are shown in Table 11 below.

TABLE 11

| | | Primers Used |
|---|---|---|
| | | (Forward Direction, |
| PCR Product | Template Used | Reverse Direction) |
| Pn-cgl aroG | c.gl 13032 genomic | SEQ ID NO: 38, |
| | DNA | SEQ ID NO: 39 |
| Pcj7-cgl aroG | c.gl 13032 genomic | SEQ ID NO: 40, |
| | DNA | SEQ ID NO: 41 |

After obtaining gene fragments using the template and primers above, each gene fragment was ligated to the pECCG 117 and pECCG 117_Pcj7_GFP_terminator (Korean Patent No. 10-620092, p117-cj7-gfp) vectors, which were treated with EcoRV/XbaI restriction enzymes, using the In-Fusion^{R} HD cloning kit (Clontech). The prepared vectors were named as pECCG117_Pn_cgl aroG and pECCG117_Pcj7_cgl aroG, respectively. The above vectors were confirmed by sequencing with regard to the success of cloning and gene sequence in each vector.

First, since a microorganism of the genus Corynebacterium cannot produce shinorine, a strain in which shinorine biosynthetic pathway is introduced was prepared. Specifically, the Ava_ABCD gene was subjected to PCR using the pECCG117_Ptrc_Ava_ABCD as a template along with a primer pair of SEQ ID NOS: 42 (forward direction) and 43 (reverse direction). The pDZTn_Ava_ABCD was prepared by ligating an about 7 kb PCR fragment to the pDZTn vector (WO 2009-125992A), which is treated with an Nde1 restriction enzyme, using the In-Fusion^{R} HD cloning kit (Clontech). Then, a fragment of the 02 promoter (KR Patent No. 10-1632642) was subjected to PCR using a primer pair of SEQ ID NOS: 44 (forward direction) and 45 (reverse direction), and ligated to the pDZTn_Ava_ABCD, which is treated with an NdeI restriction enzyme, using the In-Fusion^{R} HD cloning kit (Clontech), thereby preparing pDZTn_PO2_Ava_ABCD.

The recombinant plasmid was transformed into the wild-type ATCC13032 by electroporation (van der Rest et al. 1999), and the plasmid was introduced into the chromosome by primary recombination (cross-over), and the plasmid region was excised from the chromosome through secondary recombination (cross-over).

For each of the transformed Corynebacterium glutamicum strains, in which the secondary recombination is completed, the introduction of the Ava_ABCD gene was confirmed by PCR using a gene-specific primer pair of SEQ ID NOS: 42 (forward direction) and 43 (reverse direction). The prepared strain was named as Corynebacterium glutamicum 13032_ΔN1021PO2_Ava_ABCD.

The pECCG117 Pn_cgl aroG and pECCG117_Pcj7_cgl aroG vectors were each transformed into the Corynebacterium glutamicum 13032 ΔN1021_PO2_Ava_ABCD strain by electroporation.

The strains prepared above and the control group Corynebacterium glutamicum ATCC13032 (c.gl 13032) were cultured overnight in a BHIS solid medium containing kanamycin, and one platinum loop thereof were inoculated into a 25 mL titration medium [medium composition: glucose 40 g/L, KH₂PO₄ 1 g/L, (NH4)₂SO₄ 10 g/L, MgSO₄7H₂O 5 g/L, NaCl 5 g/L, yeast extract 5 g/L, calcium carbonate 30 g/L: pH 7.0], which was incubated in a 37° C. incubator at 200 rpm for 48 hours. The results are shown in Table 12 below.

TABLE 12

| | | Shinorine |
|---|---|---|
| | OD | Conc. |
| Name of Strain | (600 nm) | (mg/L) |
| c.gl 13032 | 72.1 | — |
| c.gl 13032ΔN1021_PO2_Ava_ABCD | 71.5 | 180 |
| c.gl 13032ΔN1021_PO2_Ava_ABCD/ | 69.6 | 250 |
| pECCG117_Pn_cgl aroG | | |
| c.gl 13032ΔN1021_PO2_Ava_ABCD/ | 71.9 | 324 |
| pECCG117_Pcj7_cgl aroG | | |

As shown in Table 12 above, when the aroG expression level was increased in the strain containing the shinorine biosynthesis gene, the concentration of shinorine was increased by 39%. In particular, when the promoter was enhanced, it was confirmed that the shinorine concentration could be improved up to 80%.

### Example 13: Preparation of Vector in which Activities of Phosphoenolpyruvate Synthetase/Transketolase are Enhanced and Evaluation of Shinorine-Producing Ability of the Same

To increase the MAAs-producing ability of a microorganism, Corynebacterium glutamicum strains in which the activity of tkt or pps is enhanced were prepared. Specifically, the tkt (transketolase; SEQ ID NO: 95 and 96) or pps (phosphoenolpyruvate synthetase; SEQ ID NOS: 97 and 98) was further introduced based on a Corynebacterium glutamicum ATCC13032 strain. The template and primers used for the preparation of the plasmid are shown in Table 13 below.

TABLE 13

| | | Primers Used |
|---|---|---|
| | | (Forward Direction, |
| PCR Product | Template Used | Reverse Direction) |
| Pn-cgl tkt | c.gl 13032 genomic DNA | SEQ ID NO: 46, |
| | | SEQ ID NO: 47 |
| Pcj7-cgl tkt | c.gl 13032 genomic DNA | SEQ ID NO: 48, |
| | | SEQ ID NO: 49 |
| Ptrc-cgl pps | c.gl 13032 genomic DNA | SEQ ID NO: 50, |
| | | SEQ ID NO: 51 |
| Pcj7-cgl pps | c.gl 13032 genomic DNA | SEQ ID NO: 52, |
| | | SEQ ID NO: 51 |

Vectors were prepared by ligating the gene fragments, which were obtained through PCR technology in which the template was matched to combination of the primers, to the pECCG117, pECCG117_Ptrc_GFP_terminator and pECCG 117_Pcj7_GFP_terminator vectors, which were treated with EcoRV/XbaI restriction enzymes, using the In-Fusion^{R} HD cloning kit (Clontech). The prepared vectors were named as pECCG117-Pn-tkt/pECCG117-Pcj7-tkt and pECCG117-Ptrc-pps/pECCG117-Pcj7-pps, respectively. The above vectors were confirmed by sequencing with regard to the success of cloning and gene sequence in each vector and then transformed into a Corynebacterium glutamicum 13032 ΔN1021_PO2_Ava_ABCD strain by electroporation. Each strain was cultured in a kanamycin-containing BHIS solid medium overnight and a platinum loop thereof was inoculated into the 25 mL titration medium of Example 12, which was incubated in a 37° C. incubator at 200 rpm for 48 hours. The results are shown in Table 14 below.

TABLE 14

| | | Shinorine |
|---|---|---|
| | OD | Conc. |
| Name of Strain | (600 nm) | (mg/L) |
| c.gl 13032ΔN1021_PO2_Ava_ABCD | 73 | 175 |
| c.gl 13032ΔN1021_PO2_Ava_ABCD/ | 72 | 211 |
| pECCG117_Pn_cgl tkt | | |
| c.gl 13032ΔN1021_PO2_Ava_ABCD/ | 71.5 | 275 |
| pECCG117-Pcj7-cgl tkt | | |
| c.gl 13032ΔN1021_PO2_Ava_ABCD/ | 70.9 | 298 |
| pECCG117-Ptrc-cgl pps | | |
| c.gl 13032ΔN1021_PO2_Ava_ABCD/ | 70.2 | 302 |
| pECCG117-Pcj7-cgl pps | | |

As shown in Table 14 above, it was confirmed that when the tkt gene or pps gene was enhanced, the shinorine production was improved up to 57% or 72%, respectively.

### Example 14: Preparation of Strain in which Activities of 2-Dehydro-3-Deoxyphosphoheptonate Aldolase/Phosphoenolpyruvate Synthetase/Transketolase are Enhanced and Evaluation of the Same

In order to increase the MAAs-producing ability of a microorganism, E. coli strains in which the activities of aroG, pps, and tkt genes are enhanced were prepared, and to confirm the presence of a higher amount of MAAs production, the 3-dehydroquinate dehydratase (aroD) was further inactivated. Specifically, to enhance aroG, pps, and tkt genes, a pDZ-ΔaroD-Pcj7-aroG-Pcj7-pps-Pcj7-tktA plasmid was prepared. The templates and primers used for the preparation of the pDZ-ΔaroD-Pcj7-aroG-Pcj7-pps-Pcj7-tktA plasmid are shown in Table 15 below.

TABLE 15

| | | Primers Used |
|---|---|---|
| | | (Forward Direction, |
| PCR Product | Template Used | Reverse Direction) |
| Pcj7-aroG | pECCG117_Pcj7_cgl aroG | SEQ ID NO: 53, |
| | | SEQ ID NO: 54 |
| Pcj7-tkt | pECCG117-Pcj7-cgl tkt | SEQ ID NO: 55, |
| | | SEQ ID NO: 56 |
| Pcj7-pps | pECCG117-Pcj7-cgl pps | SEQ ID NO: 57, |
| | | SEQ ID NO: 58 |

First, in order to prepare a strain in which the aroD gene (SEQ ID NOS: 89 and 90) of Corynebacterium glutamicum is deleted, a pDZ-ΔaroD plasmid in which the open reading frame of the aroD gene is internally deleted was prepared. The internal gene deletion of the pDZ-ΔaroD plasmid was achieved by performing a cross-PCR using the genomic DNA of the Corynebacterium glutamicum ATCC 13032 strain as a template along with SEQ ID NOS: 91 and 92, and SEQ ID NOS: 93 and 94 as a pair of forward and reverse primers, followed by introducing the resulting gene fragments into the pDZ vector.

Then, each gene fragment of the aroG, pps, and tkt genes was amplified through PCR using the templates and primers shown in Table 15 above, and was then introduced into the pDZ-ΔaroD vector, cleaved with a SpeI restriction enzyme, respectively. The above two kinds of vectors were confirmed by sequencing with regard to the success of cloning and gene sequence in each vector, and then, transformed into a Corynebacterium glutamicum 13032ΔN1021_PO2_Ava_ABCD strain by electroporation. Each strain was cultured in a kanamycin-containing BHIS solid medium overnight and a platinum loop of overnight culture of each strain was inoculated into the 25 mL titration medium of Example 12, which was incubated in a 37° C. incubator at 200 rpm for 48 hours. The results are shown in Table 16 below.

TABLE 16

| | | Shinorine |
|---|---|---|
| | OD | Conc. |
| Name of Strain | (600 nm) | (mg/L) |
| c.gl 13032ΔN1021_PO2_Ava_ABCD_ΔaroD | 73 | 425 |
| c.gl 13032ΔN1021_PO2_Ava_ABCD_ΔaroD, | 69 | 531 |
| Pcj7_aroG, Pcj7_tkt, Pcj7_pps | | |

As shown in Table 16 above, the concentration of shinorine produced in the strain where the three kinds of genes (aroG, pps, and tktA) are enhanced was increased by about 25%. It was confirmed that even in the strain, where the shinorine-producing ability was increased through the deletion of the aroD gene, shinorine could be produced at a high concentration by the combination of the three kinds of genes. Additionally, it may be interpreted that when the aroD gene is further inactivated in the strain where the three kinds of genes are combined, shinorine can be produced at an even higher concentration.

<Preparation of Yeast-Based Recombinant Microorganism Producing MAAs and Production of MAAs Using the Same>

### Example 15: Preparation of Saccharomyces cerevisiae (S. cerevisiae) Strain Producing Shinorine

In order to use a Saccharomyces cerevisiae (S. cerevisiae) strain as a strain producing shinorine, a shinorine biosynthesis gene derived from A. variabilis ATCC29413 was introduced into a vector for yeast expression. Ava_A and Ava_B genes were inserted into the pRS-413 vector using the GPD promoter. Specifically, pGPD-Ava_A and pGPD-Ava_B regions were ligated by overlapping PCR. The vectors and PCR products were treated with BamHI and SalI restriction enzymes, and then ligated using T4 ligase to prepare the pRS-413-pGPD-Ava_A-pGPD-Ava_B vector.

Then, Ava_A and Ava_B genes were inserted into the pRS-414 vector using the GPD promoter. Specifically, pGPD-Ava_C and pGPD-Ava_D regions were ligated by overlapping PCR, and then, the vectors and PCR products were treated with BamHI and SalI and ligated using T4 ligase to prepare the pRS-414-pGPD-Ava_C-pGPD-Ava_D vector. The primers and template DNAs used for the preparation of the vector are shown in Table 17 below.

TABLE 17

| | | SEQ ID NO |
|---|---|---|
| | | (Forward Direction, |
| PCR Product | Template Used | Reverse Direction) |
| pGPD | S. cerevisiae gDNA | SEQ ID NO: 59, |
| | | SEQ ID NO: 60 |
| Ava_A | A. variabilis | SEQ ID NO: 61, |
| | ATCC29413 gDNA | SEQ ID NO: 62 |
| pGPD-Ava_A | PCR product | SEQ ID NO: 59, |
| | | SEQ ID NO: 62 |
| pGPD | S. cerevisiae gDNA | SEQ ID NO: 63, |
| | | SEQ ID NO: 64 |
| Ava_B | A. variabilis | SEQ ID NO: 65, |
| | ATCC29413 gDNA | SEQ ID NO: 66 |
| pGPD-Ava_B | PCR product | SEQ ID NO: 63, |
| | | SEQ ID NO: 66 |
| pGPD-Ava_A- | PCR product | SEQ ID NO: 59, |
| pGPD-Ava_B | | SEQ ID NO: 66 |
| pGPD | S. cerevisiae gDNA | SEQ ID NO: 67, |
| | | SEQ ID NO: 68 |
| Ava_C | A. variabilis | SEQ ID NO: 69, |
| | ATCC29413 gDNA | SEQ ID NO: 70 |
| pGPD-Ava_C | PCR product | SEQ ID NO: 67, |
| | | SEQ ID NO: 70 |
| pGPD | S. cerevisiae gDNA | SEQ ID NO: 71, |
| | | SEQ ID NO: 72 |
| Ava_D | A. variabilis | SEQ ID NO: 73, |
| | ATCC29413 gDNA | SEQ ID NO: 74 |
| pGPD-Ava_D | PCR product | SEQ ID NO: 71, |
| | | SEQ ID NO: 74 |
| pGPD-Ava_C- | PCR product | SEQ ID NO: 67, |
| pGPD-Ava_D | | SEQ ID NO: 74 |

The pRS-413-pGPD-Ava_A-pGPD-Ava_B vector and the pRS-414-pGPD-Ava_C-pGPD-Ava_D vector were introduced into Saccharomyces cerevisiae CEN.PK-1D (S. cerevisiae CEN.PK-1D) strain through lithium acetate transformation, and then the presence of shinorine production was confirmed. The strain was plated on a synthetic complete (SC) solid medium, in which Trp and His (i.e., auxotrophic markers) are excluded, and cultured overnight in a 30° C. incubator. One platinum loop of the strain cultured overnight in the synthetic complete (SC) solid medium, in which Trp and His are excluded, was inoculated into the 25 mL titration medium of Table 18, and then was cultured in a 30° C. incubator at 150 rpm for 24 hours. The results are shown in Table 19 below.

TABLE 18

| | Conc. |
|---|---|
| | Used |
| Composition | (g/L) |
| Yeast Nitrogen Base | 6.7 |
| (YNB) without Amino Acids | |
| Amino Acid Mixtures (without Leucine, | 2 |
| Tryptophan, Histidine, Uracil) | |
| Glucose | 20 |

TABLE 19

| | 24 Hour | | Shinorine |
|---|---|---|---|
| | OD | Residual | Conc. |
| Name of Strain | (600 nm) | Saccharide | (mg/L) |
| CEN.PK-1D WT + | 11.2 | 0 | 331 |
| pRS413-pGPD-Ava_A- | | | |
| pGPD-Ava_B + | | | |
| pRS414-pGPD- | | | |
| Ava_C-pGPD-Ava_D | | | |

As a result of the experiment, it was confirmed that the wild-type Saccharomyces cerevisiae strain, not producing shinorine, produced 331 mg/L of shinorine due to the introduction of the shinorine biosynthesis gene.

### Example 16: Increase of Amount of Shinorine Production Through Enhancement of TKL1 (Transketolase) of Saccharomyces cerevisiae

In order to increase the MAAs-producing ability, a Saccharomyces cerevisiae strain in which the activity of TKL1 is enhanced was prepared. For this purpose, the expression of the TKL1 gene was enhanced by cloning the TKL1 gene (SEQ ID NOS: 110 and 123) into pRS-415-pGPD, pRS-415-pADH, and pRS-415-pTEF vectors

The GPD promoter included in the pRS-415-pGPD vector is the promoter of glyceraldehyde-3-phosphate dehydrogenase (GAPDH) and isozyme 3 (TDH3) genes, and it includes a sequence of −674 bp to −1 bp from the initiation codon of the ORF of the THD3 gene.

The ADH promoter included in the pRS-415-pADH vector is the promoter of alcohol dehydrogenase (ADH1) gene, and it includes a sequence of −1,500 bp to −1 bp from the initiation codon of the ORF of the ADH1 gene.

The TEF promoter included in the pRS-415-pTEF vector is the promoter of translational elongation factor EF-1 alpha (TEF1) gene, and it includes a sequence of −500 bp to −1 bp from the initiation codon of the ORF of the TEF1 gene.

Specifically, the TEF1 gene was subjected to PCR using the primers of Table 20 below, and the PCR products and pRS-415-pGPD, pRS-415-pADH, and pRS-415-pTEF vectors were treated with BamHI and SalI restriction enzymes, and then ligated using a T4 ligase to prepare the pRS-415-pGPD-TKL1, pRS-415-pADH-TKL1, and pRS-415-pTEF-TKL1 vectors.

TABLE 20

| | | SEQ ID NO |
|---|---|---|
| | | (Forward Direction, |
| PCR Product | Template Used | Reverse Direction) |
| TKL1 | S. cerevisiae gDNA | SEQ ID NO: 75, |
| | | SEQ ID NO: 76 |

Then, the plasmid for shinorine biosynthesis prepared in Example 15 was introduced into Saccharomyces cerevisiae CEN.PK-1D strain along with the pRS-415-pGPD-TKL1, pRS-415-pADH-TKL1, and pRS-415-pTEF-TKL1, and each resulting strain was plated on a synthetic complete (SC) solid medium, in which Trp, Ura, and His are excluded, and cultured overnight in a 30° C. incubator. One platinum loop of the strain cultured overnight in the synthetic complete (SC) solid medium, in which Trp, Ura, and His are excluded, was inoculated into a 25 mL titration medium, and then was cultured in a 30° C. incubator at 150 rpm for 24 hours. The results are shown in Table 21 below.

TABLE 21

| | 24 Hour | | Shinorine |
|---|---|---|---|
| | OD | Residual | Conc. |
| Name of Strain | (600 nm) | Saccharide | (mg/L) |
| CEN.PK-1D WT + | 11.2 | 0 | 327 |
| pRS413-pGPD- | | | |
| Ava_A-pGPD-Ava_B, | | | |
| pRS414-pGPD- | | | |
| Ava_C-pGPD-Ava_D | | | |
| CEN.PK-1D(pADH-TKL1) + | 13 | 0 | 370 |
| pRS413-pGPD- | | | |
| Ava_A-pGPD-Ava_B, | | | |
| pRS414-pGPD- | | | |
| Ava_C-pGPD-Ava_D | | | |
| CEN.PK-1D(pTEF-TKL1) + | 12.1 | 0 | 420 |
| pRS413-pGPD- | | | |
| Ava_A-pGPD-Ava_B, | | | |
| pRS414-pGPD- | | | |
| Ava_C-pGPD-Ava_D | | | |
| CEN.PK-1D(pGPD-TKL1) + | 12.5 | 0 | 610 |
| pRS413-pGPD- | | | |
| Ava_A-pGPD-Ava_B, | | | |
| pRS414-pGPD- | | | |
| Ava_C-pGPD-Ava_D | | | |

As shown in Table 21 above, it was confirmed that the amount of shinorine production was increased in the strain, in which the TKL1 gene expression is enhanced using the GPD promoter, compared to that of the WT strain. Additionally, it was further confirmed that as the strength of the promoter increased (i.e., pGPD>pTEF>pADH), the amount of shinorine production was increased.

### Example 17: Increase of Amount of Shinorine Production Through Enhancement of ARO4 (3-deoxy-D-arabino-heptulosonate-7-phosphate (DAHP) Synthase) of Saccharomyces cerevisiae

In order to increase the MAAs-producing ability, Saccharomyces cerevisiae strains in which the activity of ARO4 is enhanced were prepared. For this purpose, a strategy was employed in which the expression of the ARO4 gene was enhanced by cloning the ARO4 gene (SEQ ID NOS: 111 and 124) into pRS-415-pGPD, pRS-415-pADH, and pRS-415-pTEF vectors. Specifically, the ARO4 gene was subjected to PCR using the primers of Table 22 below, and the PCR products of ARO4 and pRS-415-pGPD, pRS-415-pADH, and pRS-415-pTEF vectors were treated with BamHI and SalI restriction enzymes, and then ligated using T4 ligase to prepare the pRS-415-pGPD-ARO4, pRS-415-pADH-ARO4, and pRS-415-pTEF-ARO4 vectors.

TABLE 22

| | | SEQ ID NO |
|---|---|---|
| | | (Forward Direction, |
| PCR Product | Template Used | Reverse Direction) |
| ARO4 | S. cerevisiae gDNA | SEQ ID NO: 77, |
| | | SEQ ID NO: 78 |

Then, the plasmid for shinorine biosynthesis prepared in Example 15 was introduced into Saccharomyces cerevisiae CEN.PK-1D strain along with the pRS-415-pGPD-ARO4, pRS-415-pADH-ARO4, and pRS-415-pTEF-ARO4, and each resulting strain was plated on a synthetic complete (SC) solid medium, in which Trp, Ura, and His are excluded, and cultured overnight in a 30° C. incubator. One platinum loop of the strain cultured overnight in the synthetic complete (SC) solid medium, in which Trp, Ura, and His are excluded, was inoculated into a 25 mL titration medium, and then was cultured in a 30° C. incubator at 150 rpm for 24 hours. The results are shown in Table 23 below.

TABLE 23

| | 24 Hour | | Shinorine |
|---|---|---|---|
| | OD | Residual | Conc. |
| Name of Strain | (600 nm) | Saccharide | (mg/L) |
| CEN.PK-1D WT + | 11.2 | 0 | 310 |
| pRS413-pGPD- | | | |
| Ava_A-pGPD-Ava_B, | | | |
| pRS414-pGPD- | | | |
| Ava_C-pGPD-Ava_D | | | |
| CEN.PK-1D(pADH-ARO4) + | 13 | 0 | 415 |
| pRS413-pGPD- | | | |
| Ava_A-pGPD-Ava_B, | | | |
| pRS414-pGPD- | | | |
| Ava_C-pGPD-Ava_D | | | |
| CEN.PK-1D(pTEF-ARO4) + | 12.1 | 0 | 610 |
| pRS413-pGPD- | | | |
| Ava_A-pGPD-Ava_B, | | | |
| pRS414-pGPD- | | | |
| Ava_C-pGPD-Ava_D | | | |
| CEN.PK-1D(pGPD-ARO4) + | 12.5 | 0 | 890 |
| pRS413-pGPD- | | | |
| Ava_A-pGPD-Ava_B, | | | |
| pRS414-pGPD- | | | |
| Ava_C-pGPD-Ava_D | | | |

As shown in Table 23 above, it was confirmed that the amount of shinorine production was increased by 187% in the strain, in which the ARO4 gene expression is enhanced using the GPD promoter, compared to that of the WT strain.

### Example 18: Increase of Amount of Shinorine Production Through Enhancement of Phosphoenolpyruvate Synthetase (Pps) of Saccharomyces cerevisiae

In order to increase the MAAs-producing ability, Saccharomyces cerevisiae strains in which the activity of pps is enhanced were prepared. For this purpose, a strategy was employed in which the expression of the pps gene was enhanced by cloning the pps gene into pRS-415-pGPD, pRS-415-pADH, and pRS-415-pTEF vectors and the expression of the pps gene was enhanced.

Specifically, the pps gene was subjected to PCR using the primers of Table 24 below, and the PCR products of pps and pRS-415-pGPD, pRS-415-pADH, and pRS-415-pTEF vectors were treated with BamHI and SalI restriction enzymes, and then ligated using T4 ligase to prepare the pRS-415-pGPD-pps, pRS-415-pADH-pps, and pRS-415-pTEF-pps vectors.

TABLE 24

| | | SEQ ID NO |
|---|---|---|
| | | (Forward Direction, |
| PCR Product | Template Used | Reverse Direction) |
| pps | E. coli MG1655 gDNA | SEQ ID NO: 79, |
| | | SEQ ID NO: 80 |

Then, the plasmid for shinorine biosynthesis prepared in Example 15 was introduced into Saccharomyces cerevisiae CEN.PK-1D strain along with the pRS-415-pGPD-pps, pRS-415-pADH-pps, and pRS-415-pTEF-pps, and each resulting strain was plated on a synthetic complete (SC) solid medium, in which Trp, Ura, and His are excluded, and cultured overnight in a 30° C. incubator. One platinum loop of the strain cultured overnight in the synthetic complete (SC) solid medium, in which Trp, Ura, and His are excluded, was inoculated into a 25 mL titration medium, and then was cultured in a 30° C. incubator at 150 rpm for 24 hours. The results are shown in Table 25 below.

TABLE 25

| | 24 Hour | | Shinorine |
|---|---|---|---|
| | OD | Residual | Conc. |
| Name of Strain | (600 nm) | Saccharide | (mg/L) |
| CEN.PK-1D WT + | 11.2 | 0 | 340 |
| pRS413-pGPD- | | | |
| Ava_A-pGPD-Ava_B, | | | |
| pRS414-pGPD- | | | |
| Ava_C-pGPD-Ava_D | | | |
| CEN.PK-1D(pADH-pps) + | 13 | 0 | 375 |
| pRS413-pGPD- | | | |
| Ava_A-pGPD-Ava_B, | | | |
| pRS414-pGPD- | | | |
| Ava_C-pGPD-Ava_D | | | |
| CEN.PK-1D(pTEF-pps) + | 12.1 | 0 | 410 |
| pRS413-pGPD- | | | |
| Ava_A-pGPD-Ava_B, | | | |
| pRS414-pGPD- | | | |
| Ava_C-pGPD-Ava_D | | | |
| CEN.PK-1D(pGPD-pps) + | 12.5 | 0 | 580 |
| pRS413-pGPD- | | | |
| Ava_A-pGPD-Ava_B, | | | |
| pRS414-pGPD- | | | |
| Ava_C-pGPD-Ava_D | | | |

As shown in Table 25 above, it was confirmed that the amount of shinorine production was increased by 70% in the strain, in which the pps gene is overexpressed, compared to that of the WT strain. Additionally, it was further confirmed that as the strength of the promoter increased (i.e., pGPD>pTEF>pADH), the amount of shinorine production was increased.

### Example 19: Increase of Amount of Shinorine Production Through Enhancement of TKL1, Enhancement of ARO4, and Introduction of pps Gene in Saccharomyces cerevisiae Strain

Based on the results of Examples 16, 17, and 18, TKL1, ARO4, and pps (E. coli) genes were selected as effective factors which have an influence on shinorine biosynthesis in Saccharomyces cerevisiae, and an attempt was made to increase the shinorine biosynthesis through simultaneous enhancement of these three kinds of genes. For the introduction of these three kinds of genes, the pRS-415-pGPD-TKL1-pGPD-ARO4, and pRS-416-pGPD-pps vectors were prepared. Specifically, after pGPD-TKL1 and pGPD-ARO4 regions were connected by overlapping PCR, the vectors and PCR products were treated with BamHI and SalI restriction enzymes, and then ligated using T4 ligase to prepare the pRS-415-pGPD-TKL1-pGPD-ARO4 vector.

Then, the pps gene derived from E. coli was subjected to PCR. The PCR products of the pps gene and the pRS-416-pGPD vector were treated with BamHI and SalI restriction enzymes, and then ligated using T4 ligase to prepare the pRS-416-pGPD-pps vector. The primers and template DNAs used for the preparation of the vectors are shown in Table 26 below.

TABLE 26

| | | SEQ ID NO |
|---|---|---|
| | | (Forward Direction, |
| PCR Product | Template Used | Reverse Direction) |
| pGPD | S. cerevisiae gDNA | SEQ ID NO: 81, |
| | | SEQ ID NO: 82 |
| TKL1 | S. cerevisiae gDNA | SEQ ID NO: 83, |
| | | SEQ ID NO: 84 |
| pGPD-TKL1 | S. cerevisiae gDNA | SEQ ID NO: 81, |
| | | SEQ ID NO: 84 |
| pGPD | S. cerevisiae gDNA | SEQ ID NO: 85, |
| | | SEQ ID NO: 86 |
| ARO4 | S. cerevisiae gDNA | SEQ ID NO: 87, |
| | | SEQ ID NO: 88 |
| pGPD-ARO4 | S. cerevisiae gDNA | SEQ ID NO: 85, |
| | | SEQ ID NO: 88 |
| pGPD-TKL1- | S. cerevisiae gDNA | SEQ ID NO: 81, |
| pGPD-ARO4 | | SEQ ID NO: 88 |
| ppsA | E. coli MG1655 gDNA | SEQ ID NO: 79, |
| | | SEQ ID NO: 80 |

Then, the plasmid for shinorine biosynthesis prepared in Example 15 was introduced into Saccharomyces cerevisiae CEN.PK-1D strain along with the pRS-415-pGPD-TKL1-pGPD-ARO4 and pRS-416-pGPD-pps vectors, and each resulting strain was plated on a synthetic complete (SC) solid medium, in which Leu, Trp, Ura, and His are excluded, and cultured overnight in a 30° C. incubator. One platinum loop of the strain cultured overnight in the synthetic complete (SC) solid medium, in which Leu, Trp, Ura, and His are excluded, was inoculated into a 25 mL titration medium, and then was cultured in a 30° C. incubator at 150 rpm for 24 hours. The results are shown in Table 27 below.

TABLE 27

| | 24 Hours | | Shinorine |
|---|---|---|---|
| | OD | Residual | Conc. |
| Name of Strain | (600 nm) | Saccharide | (mg/L) |
| CEN.PK-1D WT + | 11.2 | 0 | 333 |
| pRS413-pGPD- | | | |
| Ava_A-pGPD-Ava_B, | | | |
| pRS414-pGPD- | | | |
| Ava_C-pGPD-Ava_D | | | |
| CEN.PK-1D WT + | 13.3 | 0 | 1,100 |
| pRS413-pGPD- | | | |
| Ava_A-pGPD-Ava_B, | | | |
| pRS414-pGPD- | | | |
| Ava_C-pGPD-Ava_D, | | | |
| p415-pGPD- | | | |
| TKL1-pGPD-ARO4, | | | |
| p416-pGPD-pps | | | |

As shown in Table 27 above, it was confirmed that the amount of shinorine production was significantly increased by 230% in the strain, in which the three kinds of effective genes (i.e., pps, TKL1, and ARO4) are overexpressed, compared to that of the WT strain.

In this specification, detailed descriptions of the contents that can be fully recognized and inferred by one of ordinary skill in the art of the present disclosure have been omitted. In addition to the specific embodiments described in this specification, various modifications are possible without changing the technical spirit or essential constitutions of the present disclosure. Therefore, the present disclosure can be implemented in a manner different from those specifically described and exemplified in this specification, which can be understood by one of ordinary skill in the art.

1. A microorganism producing a mycosporine-like amino acid, wherein the activity of at least one protein selected from the group consisting of 2-dehydro-3-deoxyphosphoheptonate aldolase, phosphoenolpyruvate synthetase, and transketolase is enhanced.

2. The microorganism according to claim 1, wherein the microorganism further comprises a mycosporine-like amino acid biosynthesis gene cluster.

3. The microorganism according to claim 2, wherein the mycosporine-like amino acid biosynthesis gene cluster comprises a gene encoding at least one protein selected from the group consisting of 2-demethyl 4-deoxygadusol synthase, O-methyltransferase, and a C—N ligase.

4. The microorganism according to claim 2, wherein the mycosporine-like amino acid biosynthesis gene cluster comprises a gene encoding at least one protein selected from the group consisting of non-ribosomal peptide synthetase, non-ribosomal peptide synthetase-like enzyme (NRPS-like enzyme), and D-alanine D-alanine ligase (D-Ala D-Ala ligase).

5. The microorganism according to claim 1, wherein the microorganism is a microorganism of the genus Corynebacterium or a microorganism of the genus Escherichia, or yeast.

6. The microorganism according to claim 1, wherein the mycosporine-like amino acid is at least one selected from the group consisting of mycosporine-2-glycine, palythinol, palythenic acid, deoxygadusol, mycosporine-methylamine-threonine, mycosporine-glycine-valine, palythine, asterina-330, shinorine, porphyra-334, euhalothece-362, mycosporine-glycine, mycosporine-ornithine, mycosporine-lysine, mycosporine-glutamic acid-glycine, mycosporine-methylamine-serine, mycosporine-taurine, palythene, palythine-serine, palythine-serine-sulfate, palythinol, and usujirene.

7. A method for producing a mycosporine-like amino acid, comprising:
culturing the microorganism of claim 1 in a medium; and
recovering the mycosporine-like amino acid from the cultured microorganism or medium.

8. The method according to claim 7, wherein the microorganism further comprises a mycosporine-like amino acid biosynthesis gene cluster.

9. The method according to claim 8, wherein the mycosporine-like amino acid biosynthesis gene cluster comprises a gene encoding at least one protein selected from the group consisting of 2-demethyl 4-deoxygadusol synthase, O-methyltransferase, and a C—N ligase.

10. The method according to claim 8, wherein the mycosporine-like amino acid biosynthesis gene cluster comprises a gene encoding at least one protein selected from the group consisting of non-ribosomal peptide synthetase, non-ribosomal peptide synthetase-like enzyme (NRPS-like enzyme), and D-alanine D-alanine ligase (D-Ala D-Ala ligase).

11. The method according to claim 7, wherein the microorganism is a microorganism of the genus Corynebacterium, a microorganism of the genus Escherichia, or yeast.

12. The method according to claim 7, wherein the mycosporine-like amino acid is at least one selected from the group consisting of mycosporine-2-glycine, palythinol, palythenic acid, deoxygadusol, mycosporine-methylamine-threonine, mycosporine-glycine-valine, palythine, asterina-330, shinorine, porphyra-334, euhalothece-362, mycosporine-glycine, mycosporine-ornithine, mycosporine-lysine, mycosporine-glutamic acid-glycine, mycosporine-methylamine-serine, mycosporine-taurine, palythene, palythine-serine, palythine-serine-sulfate, palythinol, and usujirene.
